(19) United States
(12) Patent Application Publication
(10) Pub. No.: US 2012/0065179 A1
(43) Pub. Date: Mar. 15, 2012

(54) PRODRUG COMPRISING BETA-KETO CARBOXYLIC ACID, BETA-KETO CARBOXYLIC ACID SALT OR BETA-KETO CARBOXYLIC ACID ESTER FOR DRUG DELIVERY

(75) Inventor: Martin Andersson, Enskede (SE)

(73) Assignee: YKI, Ytemiska Institutet AB, Stockholm (SE)

(21) Appl. No.: 13/264,582

(22) PCT Filed: Apr. 13, 2010
(86) PCT No.: PCT/SE2010/050395

§ 371 Date: Nov. 22, 2011

Related U.S. Application Data
(60) Provisional application No. 61/169,028, filed on Apr. 14, 2009.

(51) Int. Cl. A61K 31/568; C07J 1/00; C07C 59/84; A61K 31/216; A61K 31/192; C07C 51/09; C07C 69/738; C07C 59/90

(52) U.S. Cl. 514/179; 560/53; 552/635; 562/462; 514/510; 514/569; 560/51; 562/459; 514/545; 514/570

(57) ABSTRACT

There is provided a prodrug of a pharmaceutically active agent, such prodrug comprising a beta-keto carboxylic acid, a beta-keto carboxylic acid salt or a beta-keto carboxylic acid ester functional group, a pharmaceutical composition comprising the prodrug, and to the use of the prodrug or composition for treatment of a mammalian subject suffering from a condition which can be cured or alleviated by administration of the pharmaceutically active agent. There is further provided a method of inhibiting decarboxylation of a compound comprising a beta-keto carboxylic acid or a salt thereof with a monovalent cation, characterized in that a dry salt of the beta-keto carboxylic acid with a divalent or polyvalent cation is prepared.

### FIELD OF THE INVENTION

The present invention relates to novel prodrugs of pharmaceutically active agents, pharmaceutical compositions comprising such prodrugs, and uses thereof, wherein said prodrugs provide improved aqueous solubility, sustained release and/or improved bioavailability of said pharmaceutically active agents.

### BACKGROUND

The term “prodrug”, as used in the present specification, relates to a derivative of a known and proven organic pharmaceutically active agent, wherein said derivative, when administered to a warm blooded animal, such as a human, is converted into the pharmaceutically active agent. Conversion of the derivative may occur through a number of different mechanisms involving e.g. chemical and/or enzymatic reactions. Often, the conversion of the prodrug comprises the cleavage of one or more chemical bonds, resulting in the formation of two or more cleavage products, at least one of said cleavage products being the pharmaceutically active agent and the other being non-toxic or metabolizes to form non-toxic metabolites.

Development of prodrugs is an established strategy for improving physicochemical, biopharmaceutical and pharmacokinetic properties of pharmacologically potent compounds for use in drug compositions.

Technologies such as high throughput screening and combinatorial chemistry commonly used in drug discovery often produce novel lead structures having high pharmacological potency, but lacking suitable physicochemical, biopharmaceutical and pharmacokinetic properties for use in drug compositions.

As an example, approximately 40% of the drug candidates produced from combinatorial screening programs have poor aqueous solubility, i.e. an aqueous solubility of less than 10 μM (Rautio et al., Nature Reviews Drug Discovery, Vol. 7, 2008). Low aqueous solubility of a pharmaceutically active agent may limit its clinical use since it may be difficult or impossible to administer a therapeutically relevant dose of the pharmaceutically active agent to the patient.

Furthermore, many known and proven pharmaceutically active agents suffer from low retention time in vivo due to rapid degradation and excretion of the agent upon administration. In order to maintain a therapeutically active concentration of such a pharmaceutically active agent in a patient, administration several times daily may be required. Optionally, a slow or controlled release device or composition may be used, which administrates the pharmaceutically active agent continuously in a slow or controlled manner.

Many different functional groups are used as the cleavable group in prodrugs. Esters are the most common prodrugs used and a majority of all marketed prodrugs are based on activation by enzymatic hydrolysis (Rautio, et al.). Ester bonds are readily hydrolyzed in vivo, e.g. by esterases found in the blood, liver and other organs and tissues, resulting in the formation of an alcohol and a carboxylic acid. However, accurate prediction of the release of a pharmaceutically active agent from an ester based prodrug is difficult since esterase activity may vary significantly between species (Rautio, et al.).

### SUMMARY OF THE INVENTION

It is an object of the present invention to solve or alleviate at least some of the above mentioned problems.

It is an object of the present invention to provide a prodrug which is converted in vivo to a pharmaceutically active agent comprising a ketone functional group.

It is an object of the present invention to provide a prodrug which is converted in vivo over a prolonged period of time to form a pharmaceutically active agent.

It is another object of the invention to provide a prodrug of a pharmaceutically active agent, wherein the conversion of said prodrug in vivo is not dependent on high or low pH values or any specific enzymatic reaction in order to produce the desired pharmaceutically active agent.

It is a further object of the present disclosure to provide a prodrug of a pharmaceutically active agent which gives sustained release and/or improved bioavailability and/or improved aqueous solubility of the pharmaceutically active agent when it is administered in the form of the prodrug.

The above objects, as well as other objects that will be apparent to a person skilled in the art in view of the present disclosure, are achieved by the present invention through the provision of a prodrug comprising a beta-keto carboxylic acid, a beta-keto carboxylic acid salt or a beta-keto carboxylic acid ester functional group, for use in therapy.

The prodrug of the invention comprising a beta-keto carboxylic acid, a beta-keto carboxylic acid salt or a beta-keto carboxylic acid ester functional group may preferably have the general formula (I)

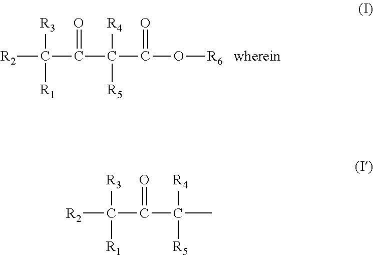

represents a residue of a pharmaceutically active agent having the general formula

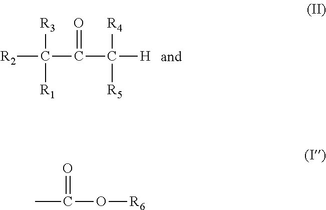

represents —COOH, a salt of —COOH with a physiologically acceptable cation, or an ester of —COOH.

The present invention is based on the inventive realization that a derivative of a pharmaceutically active agent, said derivative comprising a beta-keto carboxy functional group, may be useful as a prodrug for sustained release and/or improved bioavailability and/or improved aqueous solubility of the pharmaceutically active agent. A derivative of a pharmaceutically active agent comprising at least one ketone group may be prepared by the introduction of a functional group comprising a carboxylic acid, or a salt or an ester thereof, at a position in said pharmaceutically active agent such that a beta-keto carboxylic acid, or a salt or an ester thereof, is formed together with a ketone group of said pharmaceutically active agent. The compound thus formed comprises a beta-keto carboxy functional group. Such a derivative comprising a beta-keto carboxylic acid or a salt or an ester thereof may act as a prodrug of the pharmaceutically active agent, which will decompose in vivo upon administration to a subject to form the pharmaceutically active agent.

Thus, the prodrugs of the present disclosure may be useful as prodrugs for the sustained release of various proven pharmaceutically active agents comprising a ketone group in their chemical structure. The cleavage products are physiologically acceptable and non-toxic at therapeutically relevant concentrations of the prodrug.

The prodrug of the present disclosure may, in different embodiments thereof, provide a number of different desirable properties to the pharmaceutically active agent. Properties that may be provided by a prodrug according to the present disclosure include, but are not limited to, aqueous solubility, hydrophilicity and lipophilicity.

The prodrugs of the present disclosure may comprise one or more beta-keto carboxy functional groups. A prodrug according to the present disclosure wherein the pharmaceutically active agent comprises one ketone group may comprise between one and four carboxy functional groups bound to the alpha-carbon atoms of the ketone group. Preferably, only one carboxy functional group is bound to the alpha-carbon atom of the ketone group. In a prodrug of a pharmaceutically active agent comprising more than one ketone group, one or more of said ketone groups may be converted to beta-keto carboxy functional groups in said prodrug. A prodrug according to the present disclosure wherein the pharmaceutically active agent comprises more than one ketone group may comprise between one and four carboxy functional groups bound to the alpha-carbon atoms of each ketone group. Preferably, only one carboxy functional group is bound to the alpha-carbon atom of each ketone group.

In an embodiment of the prodrug of the present disclosure, formula (I″) represents —COOH or a salt of —COOH with a physiologically acceptable cation.

Beta-keto carboxylic acid and salt groups undergo spontaneous thermal decomposition. Decomposition is generally accelerated when the groups are dissolved in water. During decomposition of a prodrug according to the present disclosure, the carboxylic acid group is split off to form carbon dioxide. This decarboxylation is temperature dependent and the decomposition does not rely upon the presence of high or low pH or any specific enzymatic reactions in order to produce the desired pharmaceutically active agent. The decomposition may occur at predictable reaction rates at physiologically relevant temperatures, such as at 37° C. or even lower. The decarboxylation generally results in full conversion of the prodrug.

The promoiety, i.e. the group which is released from the prodrug during formation of the pharmaceutically active agent, is carbon dioxide (CO₂), hydrogen carbonate (HCO₃⁻) or carbonate (CO₃⁻) depending on the surrounding pH. CO₂, HCO₃⁻ and CO₃⁻ are non-toxic metabolites that occur naturally in the human and animal body. This is an important advantage of the prodrug of present invention as compared to prior art prodrugs for the formation of ketone containing pharmaceutically active agents. For example, oximes and imines are chemical groups often used in prodrugs for ketone containing pharmaceutically active agents (Rautio et al.). Upon hydrolysis, oximes form hydroxylamine, which has a LD₅₀ value of 192 mg/kg of body weight in rat. Likewise, upon hydrolysis, imines form amines, such as methyl amine (LD₅₀ value of 100 mg/kg in rat) or ethyl amine (LD₅₀ value of 280 mg/kg in rat). The corresponding LD₅₀ value in rat for the promoiety of the prodrugs of the present disclosure comprising a beta-keto carboxylic acid or salt group is 4220 mg/kg (based on NaHCO₃). The promoiety of the prodrugs of the present disclosure comprising a beta-keto carboxylic acid or salt group is thus significantly less toxic than the promoieties of the prior art prodrugs for ketone containing pharmaceutically active agents.

Many of the prodrugs known from the prior art rely on enzymatic cleavage of ester bonds. The enzymatic bioconversion of esters may be slow and incomplete in human blood, which may result in lower bioavailability than predicted. The cleavage of the beta-keto carboxylic acid or salt group of the prodrug of the present disclosure does not depend on enzymatic activity and will proceed to full conversion in human blood regardless of the presence of enzymatic activity.

Prodrugs based on enzymatic cleavage of a bonds to produce the pharmaceutically active agent may be sensitive to sterical hindrance of the bond to be cleaved. An advantage of the prodrugs of the present disclosure comprising a beta-keto carboxylic acid or salt group is that, since they do not rely upon any enzymatic cleavage of bonds, they may be less sensitive to sterical hindrance of the bond to be cleaved than prodrugs relying on enzymatic cleavage.

Prodrugs of the present disclosure comprising a beta-keto carboxylic acid or salt group will generally exhibit higher aqueous solubility and/or higher dissolution rate as compared to their corresponding pharmaceutically active agent. Prodrugs of the present disclosure comprising a beta-keto carboxylic acid salt have been shown to have especially high aqueous solubility. Furthermore, prodrugs of the present disclosure comprising a beta-keto carboxylic acid or salt group will generally exhibit higher aqueous solubility compared to known prior art prodrugs for ketone containing pharmaceutically active agents comprising oximes or imines.

Prodrugs of the present disclosure comprising a beta-keto carboxylic acid or salt group may be especially useful for improving the oral and/or parenteral availability of a pharmaceutically active agent. It is known in the art (e.g. from Rautio et al.) that oral and/or parenteral availability of a pharmaceutically active agent may be improved by enhancing its aqueous solubility. This may be achieved by the introduction of an ionizable group, such as an acid or a salt thereof.

The inherent labile nature of beta-keto carboxylic acids and salts thereof has meant that compounds comprising such groups have not previously been contemplated for use in pharmaceutical compositions. Since many compounds comprising beta-keto carboxylic acids and salts thereof will decompose spontaneously under ambient conditions, there may be difficulties associated with handling and storing the compounds prior to use. The present inventor has surprisingly realized that the rate of decomposition of the prodrugs of the present disclosure may be reduced by simple means to obtain prodrugs having acceptable storage and handling properties.

In an embodiment, the prodrug comprises a beta-keto carboxylic acid salt. The beta-keto carboxylic acid salt preferably comprises a cation which is physiologically acceptable and non-toxic at relevant therapeutic concentrations. The cation may be monovalent, such as Na⁺, K⁺, NH₄⁺, mono-, di- or triethanolammonium, mono-, di- or trialkylammonium, protonated forms of lysine or arginine, or divalent, such as Ca²⁺, Mg²⁺ or Fe²⁺, or have higher valency, such as Al³⁺ or Fe³⁺. In an embodiment, the cation is selected from the group consisting of Na⁺, K⁺, NH₄⁺, mono-, di- or triethanolammonium, mono-, di- or trialkylammonium, protonated forms of lysine or arginine, Ca²⁺, Mg²⁺, Fe²⁺ or Fe³⁺. The cation may preferably be a metal ion. The cation may preferably be a divalent or trivalent metal cation.

Divalent metal ions have been found to be especially useful in the present disclosure, since they have surprisingly been found to provide additional stabilization to the beta-keto carboxylic acids of the prodrugs of the present disclosure when present in the form of the dry salts of the divalent metal ions. Therefore, the physiologically acceptable salt of the prodrug of the present disclosure may preferably be a salt of the beta-keto carboxylic acid of the prodrug with a divalent metal ion, preferably Ca²⁺, Mg²⁺ or Fe²⁺ or a mixture thereof, more preferably Ca²⁺ or Mg²⁺ or a mixture thereof. Most preferably, the cation is Ca²⁺.

The prodrug of the present disclosure has been found to be particularly stable in solid form. Thus, in an embodiment, the prodrug of the present disclosure is preferably a dry solid.

In an embodiment of the prodrug, (I″) represents an ester group. Since both the thermal decomposition of the beta-keto carboxylic acid or a salt thereof, and the hydrolysis of esters of beta-keto carboxylic acids under alkaline or acidic conditions, or by enzymatic mechanisms, occur in vivo upon administration of a prodrug comprising such groups, both prodrugs comprising beta-keto carboxylic acids or salts thereof, and esters of beta-keto carboxylic acids may be employed in the present invention.

Esters of beta-keto carboxylic acids may undergo hydrolysis under alkaline or acidic conditions, or by enzymatic mechanisms, resulting in the formation of the corresponding beta-keto carboxylic acid or a salt thereof. The formed beta-keto carboxylic acid or a salt thereof will subsequently be susceptible to thermal decomposition as described in the preceding paragraph.

Prodrugs of the present disclosure comprising a beta-keto carboxylic acid ester are hydrolyzed more rapidly under alkaline conditions than ordinary esters without a beta-keto group (Paredes et al., Bol. Soc. Chil. Quim., vol. 36, 1991, 195-201). This more rapid hydrolysis may probably be explained, at least in part, by saponification via the enol mechanism. Rapid hydrolysis of the beta-keto ester may often be desirable in order to achieve a predictable release of the pharmaceutically active agent with the decarboxylation of the beta-keto carboxylic acid as the rate-limiting step, or in order to make efficient use of the higher solubility and/or improved bioavailability of the beta-keto acid or salt as compared to the pharmaceutically active agent.

Prodrugs of the present disclosure comprising a beta-keto carboxylic acid ester may also be especially useful for pharmaceutically active agents suffering from poor bioavailability due to low permeability, e.g. intestinal permeability. It is known in the art (e.g. from Rautio et al.) that drug permeability may be enhanced by masking polar or charged moieties, e.g. by making an ester of an acid.

A prodrug of the present disclosure, which comprises an ester of the beta-keto carboxylic acid group with a physiologically acceptable compound comprising a hydroxy functional group may also be considered a pro-prodrug or a precursor for the prodrug. The pro-prodrug may be converted to the prodrug ex vivo, e.g. by hydrolysis of the ester group before the prodrug is administered to a subject, or in vivo, by hydrolysis inside the body, e.g. in the gastrointestinal tract, of the subject upon administration of the ester pro-prodrug. In the latter case, the sustained release of the pharmaceutically active agent will comprise two steps, the hydrolysis of the ester group and the subsequent decarboxylation of the beta-keto carboxylic acid or salt thereof. Each of these two steps may be rate determining for the formation of the pharmaceutically active agent.

The ester group of the prodrug of the present disclosure preferably comprises an R₆ group which is physiologically acceptable and non-toxic at relevant therapeutic concentrations.

In an embodiment, R₆ is selected from the group consisting of a physiologically acceptable C₁-C₂₀ substituted or unsubstituted alkyl group, or a residue of a physiologically acceptable polymer, selected from the group consisting of water-soluble polymers, water-dispersible polymers and water-swellable polymers, or a mixture thereof.

The R₆ group in the prodrug of the present disclosure may for example be a substituted or unsubstituted alkyl group comprising 1-20 carbon atoms, such as e.g. methyl, ethyl, propyl, isopropyl, butyl, isobutyl, sec-butyl and tert-butyl, pentyl, hexyl, heptyl, octyl, nonyl, decyl, dodecyl, isopentyl, 2-ethylbutyl, cyclohexyl, 2-cyclohexyl, glyceryl or a mixture thereof.

R₆ may preferably be methyl or ethyl, since decomposition products from methyl and ethyl esters are common metabolic by-products which may be readily taken care of by the metabolic system of a subject. R₆ may preferably be ethyl.

R₆ may also be a residue of a physiologically acceptable natural or synthetic polymer. Having a large molecule, such as a physiologically acceptable natural or synthetic polymer, as R₆ may be useful to reduce the mobility of the prodrug in vivo, e.g. if a local therapeutic effect is desired.

The physiologically acceptable polymer may be selected from water-soluble polymers, water-dispersible polymers or water-swellable polymers or any mixture thereof.

In an embodiment, R₆ is a residue of a physiologically acceptable polymer, selected from the group consisting of homopolymers and copolymers of cellulose esters and cellulose ethers, hydroxyalkylcelluloses, cellulose phthalates or succinates, polyalkylene oxides, polyvinyl alcohol, polyvinyl alcohol-polyethylene glycol-graft copolymers, oligo- and polysaccharides, or a mixture thereof.

In an embodiment, R₆ is selected so as to increase the hydrophilicity of the prodrug. Increasing the hydrophilicity may be advantageous for certain modes of administration wherein a hydrophilic character of the prodrug is beneficial.

In another embodiment, R₆ is selected so as to increase the lipophilicity of the prodrug. Increased lipophilicity may improve membrane permeability and oral absorption of the prodrug. Increased lipophilicity may e.g. be used for improving ophthalmic absorption of the prodrug or for improving access of the prodrug to the central nervous system.

Prodrugs of the present disclosure allow the hydrophilic and lipophilic properties of the pharmaceutically active agent to be tailored by selection of an appropriate beta-keto carboxylic group, i.e. acid, or a suitable salt or ester. An optimal balance of hydrophilic and lipophilic properties may often be an important factor for drug permeation.

The physiologically acceptable ester of the prodrug of the present disclosure may comprise a cyclic beta-keto ester, wherein R₆ constitutes a part of the residue of the pharmaceutically active agent, and such that the prodrug forms a cyclic beta-keto ester. Ring opening of such a cyclic beta-keto ester will result in the formation of a beta-keto carboxylic acid, further comprising a hydroxyl group. Subsequent thermal decarboxylation of the formed beta-keto carboxylic acid will result in the formation of the proven pharmaceutically active agent, of which R₆ constitutes a part. Thus, in an embodiment of the prodrug, R₆ constitutes a part of said residue of a pharmaceutically active agent, such that the prodrug comprises a cyclic beta-keto carboxylic ester.

In another aspect thereof, the present disclosure provides a precursor for a prodrug of a proven pharmaceutically active agent having the general formula

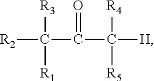

said precursor comprising an alkyl ketene dimer group arranged to form a prodrug comprising a beta-keto carboxylic acid upon hydrolysis. An example of such a prodrug precursor is

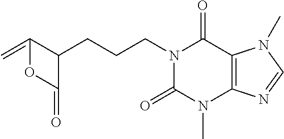

which decomposes by hydrolysis followed by decarboxylation to form the pharmaceutically active agent pentoxifylline.

The terms “ketone group”, “ketone” and “keto”, as used in the present specification, refer to an organic functional group comprising an oxygen atom attached to a first carbon atom by a double bond, and said first carbon atom being directly attached to a second and a third carbon atom by two single bonds.

The “pharmaceutically active agent” of the present invention should comprise at least one ketone functional group in its chemical structure. This ketone functional group constitutes the “beta-keto” moiety of the beta-keto carboxylic acid, or a salt or an ester thereof, in the prodrug of the present disclosure. Decarboxylation of the beta-keto carboxylic acid or salt group of a prodrug according to the invention will result in the formation of the pharmaceutically active agent comprising a ketone group.

The pharmaceutically active agent of the prodrug of the present disclosure may be any pharmaceutically active agent comprising a ketone functional group, and being susceptible to chemical modification whereby the pharmaceutically active agent is converted to a beta-keto carboxylic acid, or a salt or an ester thereof. The ketone group of the pharmaceutically active agent should preferably be arranged such that the pharmaceutically active agent may be modified to arrive at a product comprising a beta-keto carboxy functional group, in which the mentioned ketone group constitutes the beta-keto moiety. Such pharmaceutically active agents may readily be recognized by a person skilled in organic chemistry.

Examples of proven pharmaceutically active agents suitable for use in a prodrug according to the present disclosure include, but are not limited to, Alclometasone, Alprostadil, Beclometasone, Betamethasone, Boceprevir, Budesonide, Bupropion, Camphor, Clarithromycine, Clobetasol, Clobetasone, Cortisone, Cyproterone, Daunomycin, Desonide, Desoximetasone, Dexamethasone, Dinoprostone, Docetaxel, Donepezil, Doxorubicin, Droperidol, Dydrogesterone, Ebastine, Epirubicin, Equilin, Erythromycin, Estrone, Etonogestrel, Everolimus, Exemestane, Fludrocortisone, Flumetasone, Fluocinolone acetonide, Fluprednidene, Gemeprost, Haloperidol, Hydrocortisone, Hydromorphone, Idarubicin, Ketamine, Ketobemidone, Ketotifen, Levo Norgestrel, Lofepramine, Medroxyprogesterone, Megestrol, Melperone, Methadone, Methylprednisolone, Mifepristone, Misoprostol, Mometasone, Nabumetone, Naloxone, Naltrexone, Nandrolone, Nomegestrol, Norethisterone, Ondansetron, Oxcarbazepine, Oxycodone, Paclitaxel, Patupilone, Pentoxifylline, Prednisolone, Prednisone, Progesterone, Propafenone, Propiomazine, Quinupristine, Rimexolone, Sirolimus, Sitaxentan, Spironolactone, Tacrolimus, Testosterone, Tibolone, Triamcinolone, Trimegestone and Warfarin.

In an embodiment, said pharmaceutically active agent is selected from the group consisting of Alclometasone, Camphor, Clarithromycine, Clobetasone, Cyproterone, Daunomycin, Desoximetasone, Droperidol, Dydrogesterone, Ebastine, Erythromycin, Haloperidol, Idarubicin, Ketobemidone, Medroxyprogesterone, Megestrol, Melperone, Methadone, Nabumetone, Pentoxifylline, Progesterone, Propafenone, Propiomazine, Rimexolone, Sirolimus, Tacrolimus, Warfarin, Boceprevir, Everolimus, Patupilone and Sitaxentan.

In another embodiment, said pharmaceutically active agent is selected from the group consisting of Alclometasone, Camphor, Clarithromycine, Clobetasone, Cyproterone, Daunomycin, Desoximetasone, Droperidol, Dydrogesterone, Ebastine, Erythromycin, Haloperidol, Idarubicin, Ketobemidone, Medroxyprogesterone, Megestrol, Melperone, Methadone, Nabumetone, Pentoxifylline, Progesterone, Propafenone, Propiomazine, Rimexolone, Sirolimus, Tacrolimus and Warfarin.

In another embodiment, said pharmaceutically active agent is selected from the group consisting of Alclometasone, Camphor, Clobetasone, Cyproterone, Daunomycin, Desoximetasone, Droperidol, Dydrogesterone, Ebastine, Haloperidol, Idarubicin, Ketobemidone, Medroxyprogesterone, Megestrol, Melperone, Methadone, Nabumetone, Pentoxifylline, Progesterone, Propafenone, Propiomazine, Rimexolone, Tacrolimus and Warfarin.

In another embodiment, said pharmaceutically active agent is Nabumetone.

The prodrug of the present disclosure may be, but is not limited to, a compound selected from the following group consisiting of compounds listed in Table 1, wherein R₆ is as defined above.

TABLE 1
Prodrugs

| Compound | | Pharmaceutically |
|---|---|---|
| Id No. | Prodrug structure | active agent |
| 1 | 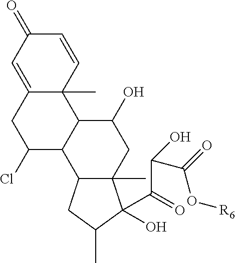 | Alclometasone |
| 2 | 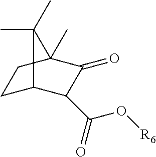 | Camphor |
| 3 | 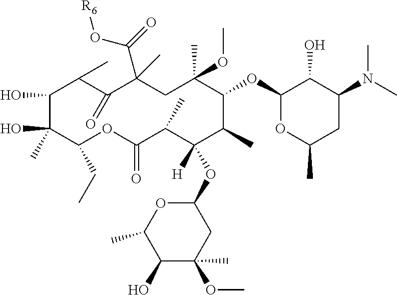 | Clarithromycine |
| 4 | 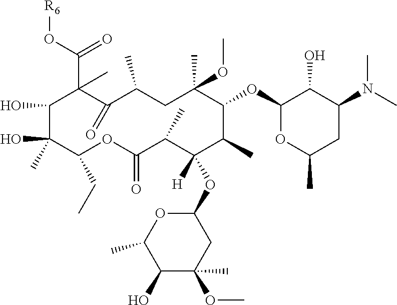 | Clarithromycine |
| 5 | 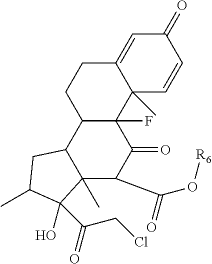 | Clobetasone |
| 6 | 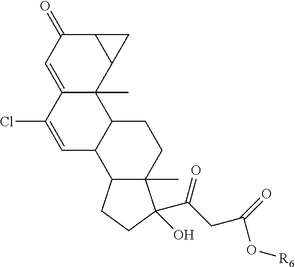 | Cyproterone |
| 7 | 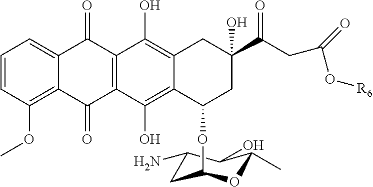 | Daunomycin |
| 8 | 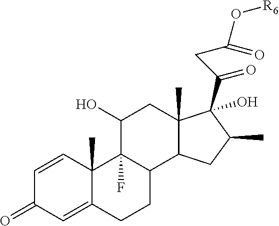 | Desoximetasone |
| 9 | 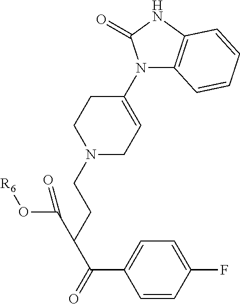 | Droperidol |
| 10 | 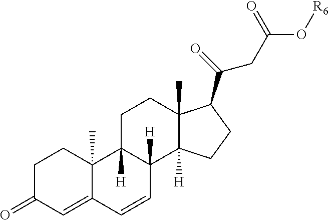 | Dydrogesterone |
| 11 | 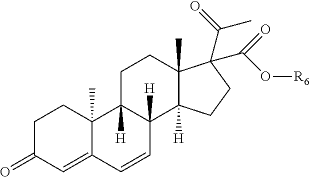 | Dydrogesterone |
| 12 | 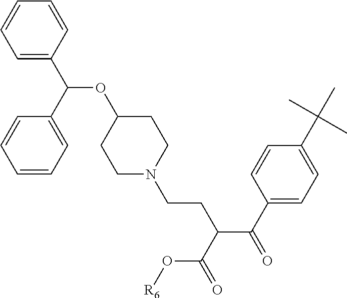 | Ebastine |
| 13 | 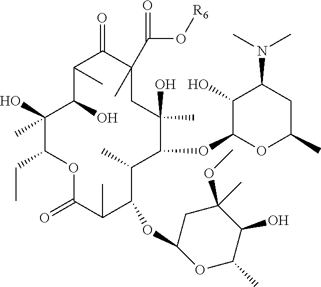 | Erythromycin |
| 14 | 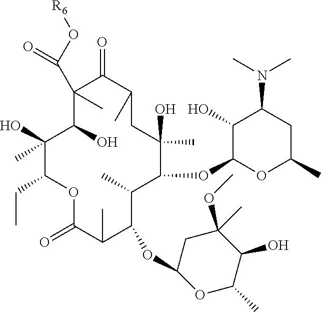 | Erythromycin |
| 15 | 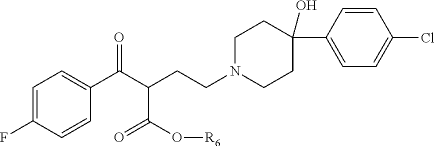 | Haloperidol |
| 16 | 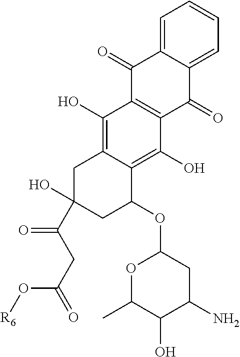 | Idarubicin |
| 17 | 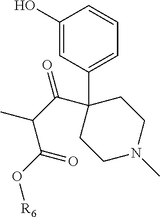 | Ketobemidone |
| 18 | 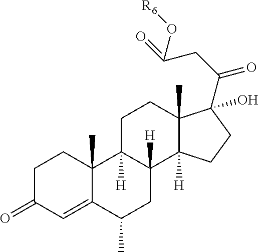 | Medroxyprogesterone |
| 19 | 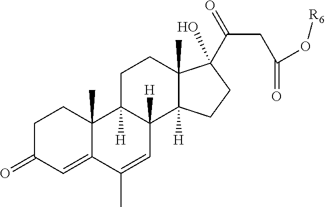 | Megestrol |
| 20 | 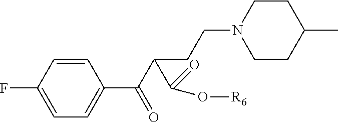 | Melperone |
| 21 | 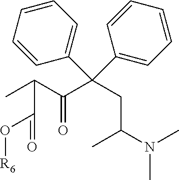 | Methadone |
| 22 | 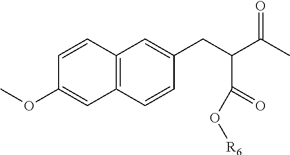 | Nabumetone |
| 23 | 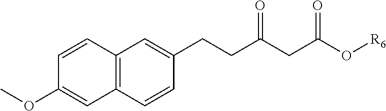 | Nabumetone |
| 24 | 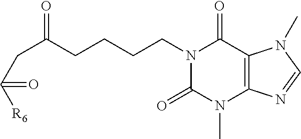 | Pentoxifylline |
| 25 | 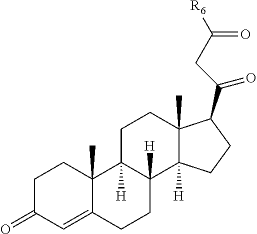 | Progesterone |
| 26 | 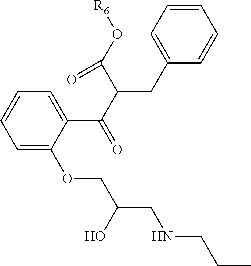 | Propafenone |
| 27 | 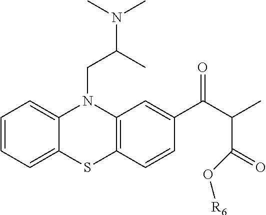 | Propiomazine |
| 28 | 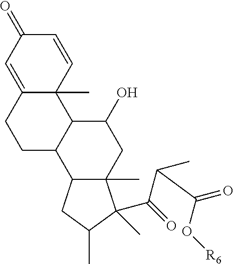 | Rimexolone |
| 29 | 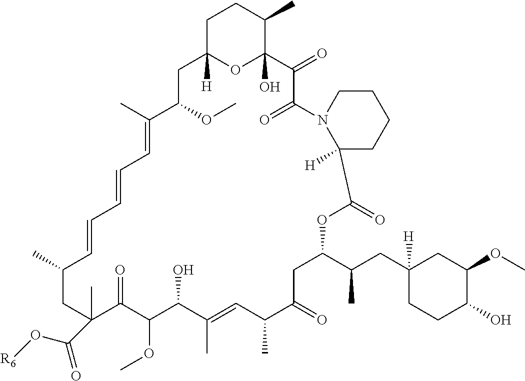 | Sirolimus |
| 30 | 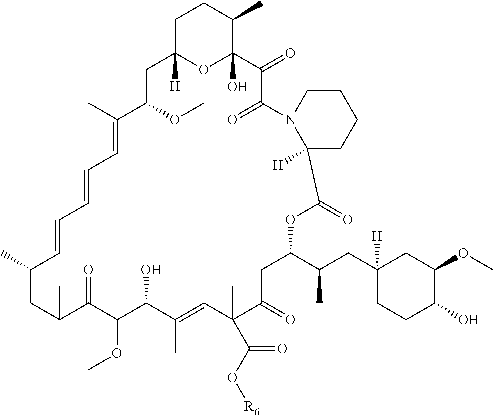 | Sirolimus |
| 31 | 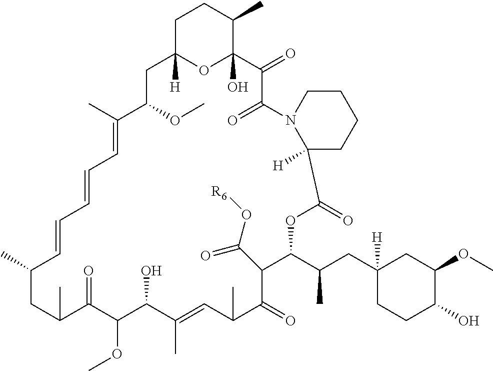 | Sirolimus |
| 32 | 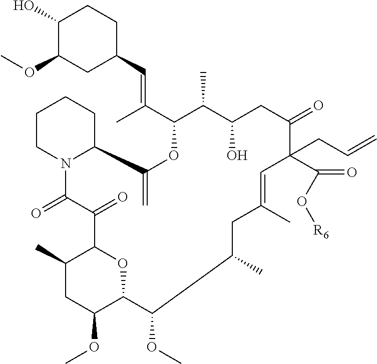 | Tacrolimus |
| 33 | 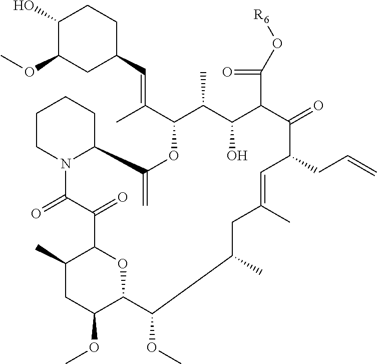 | Tacrolimus |
| 34 | 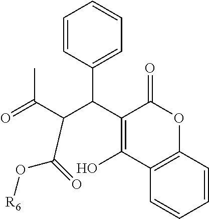 | Warfarin |
| 35 | 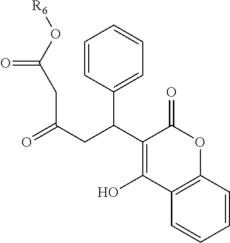 | Warfarin |
| 36 | 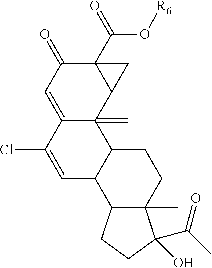 | Cyproterone |
| 37 | 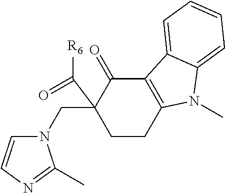 | Ondansetron |
| 38 | 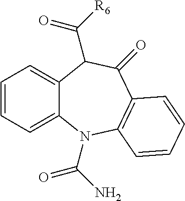 | Oxcarbazepine |
| 39 | 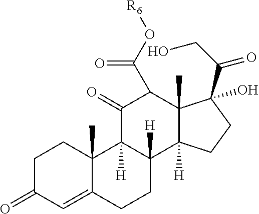 | Cortisone |
| 40 | 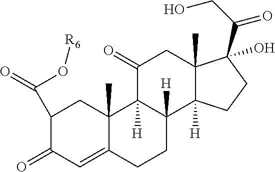 | Cortisone |
| 41 | 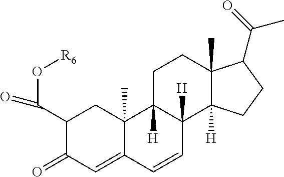 | Dydrogesterone |
| 42 | 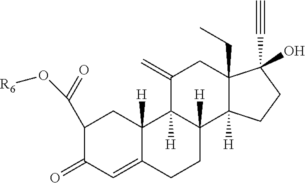 | Etonogestrel |
| 43 | 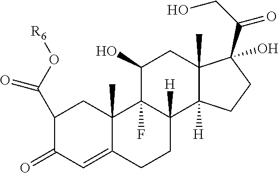 | Fludrocortisone |
| 44 | 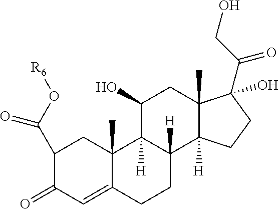 | Hydrocortisone |
| 45 | 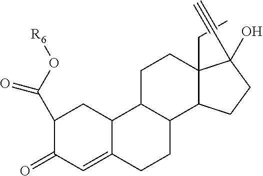 | Levo Norgestrel |
| 46 | 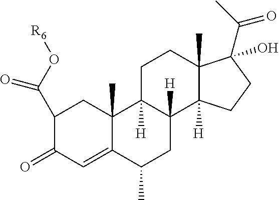 | Medroxyprogesterone |
| 47 | 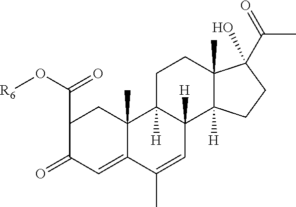 | Megestrol |
| 48 | 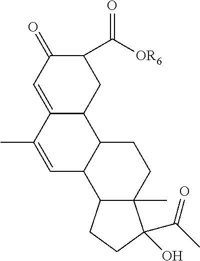 | Nomegestrol |
| 49 | 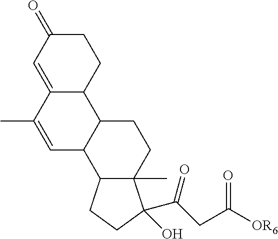 | Nomegestrol |
| 50 | 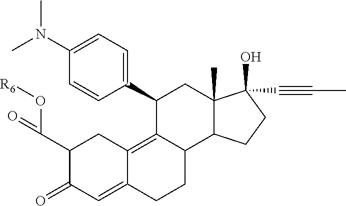 | Mifepristone |
| 51 | 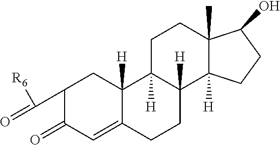 | Nandrolone |
| 52 | 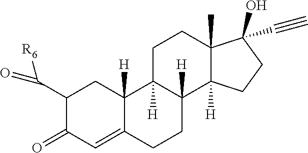 | Norethisterone |
| 53 | 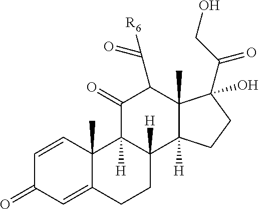 | Prednisone |
| 54 | 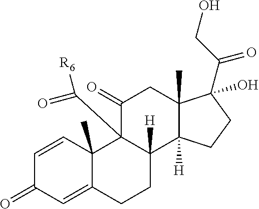 | Prednisone |
| 55 | 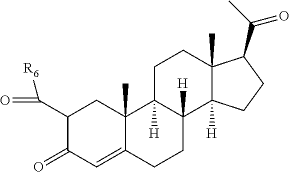 | Progesterone |
| 56 | 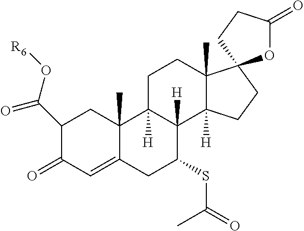 | Spironolactone |
| 57 | 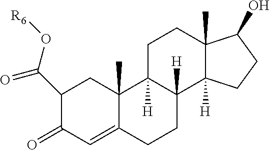 | Testosterone |
| 58 | 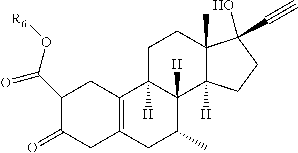 | Tibolone |
| 59 | 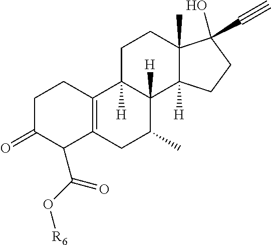 | Tibolone |
| 60 | 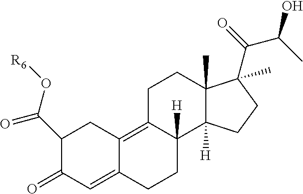 | Trimegestone |
| 61 | 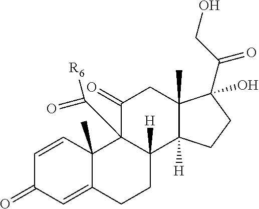 | Prednisone |
| 62 | 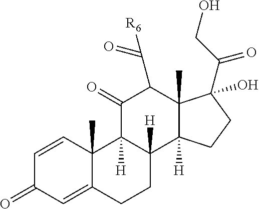 | Prednisone |
| 63 | 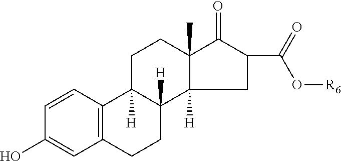 | Estrone |
| 64 | 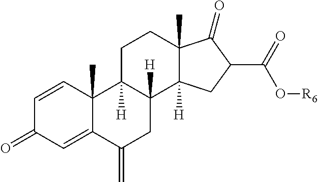 | Exemestane |
| 65 | 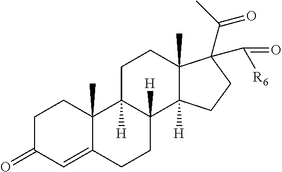 | Progesterone |
| 66 | 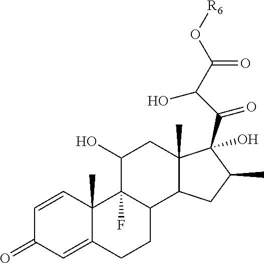 | Betamethasone |
| 67 | 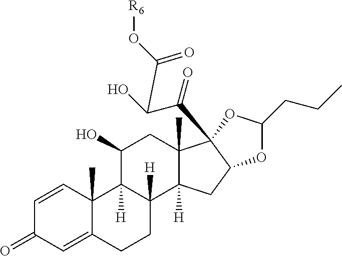 | Budesonide |
| 68 | 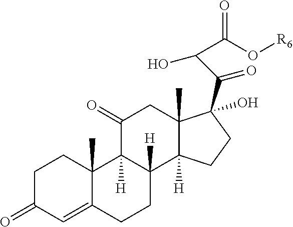 | Cortisone |
| 69 | 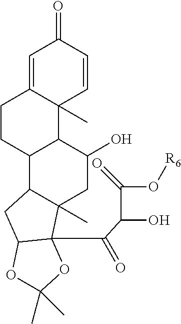 | Desonide |
| 70 | 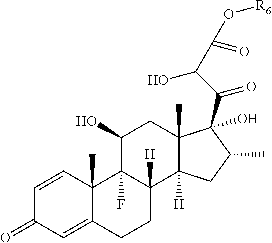 | Dexamethasone |
| 71 | 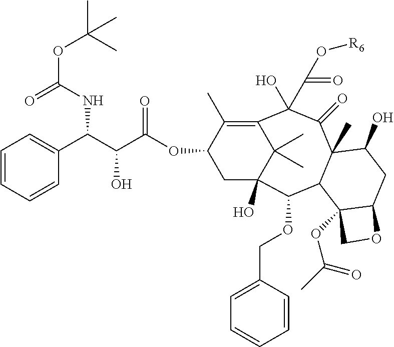 | Docetaxel |
| 72 | 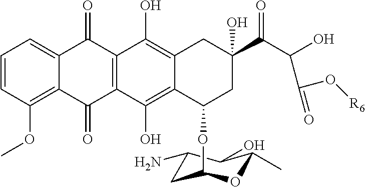 | Doxorubicin |
| 73 | 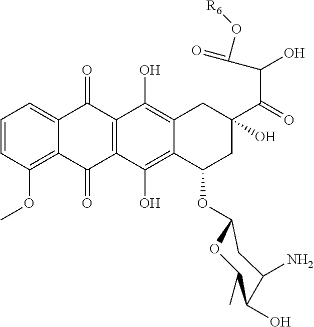 | Epirubicin |
| 74 | 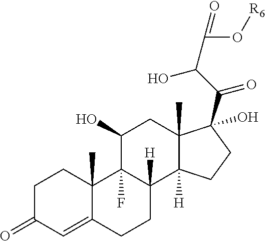 | Fludrocortisone |
| 75 | 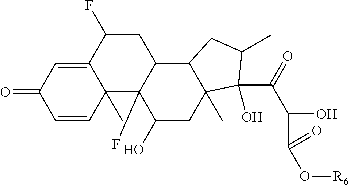 | Flumetasone |
| 76 | 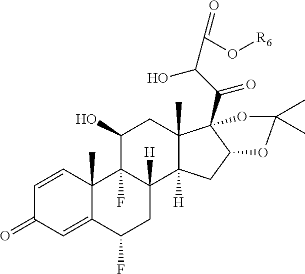 | Fluocinolone- acetonide |
| 77 | 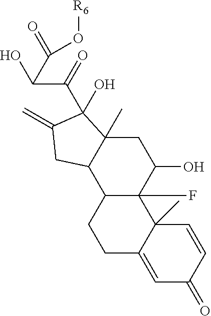 | Fluprednidene |
| 78 | 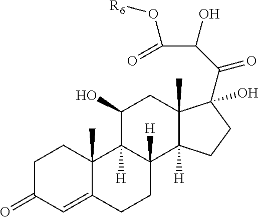 | Hydrocortisone |
| 79 | 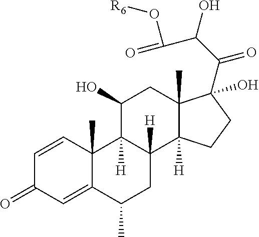 | Methylprednisolone |
| 80 | 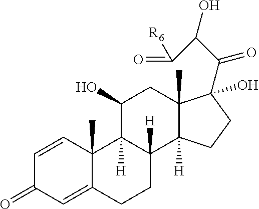 | Prednisolone |
| 81 | 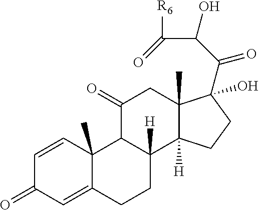 | Prednisone |
| 82 | 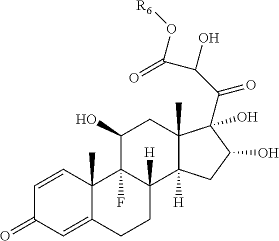 | Triamcinolone |
| 83 | 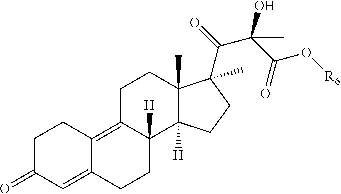 | Trimegestone |
| 84 | 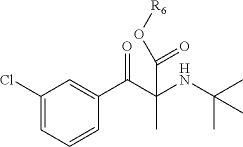 | Bupropion |
| 85 | 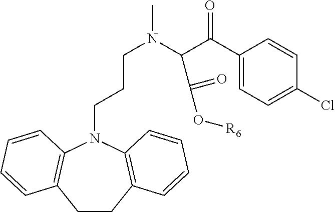 | Lofepramine |
| 86 | 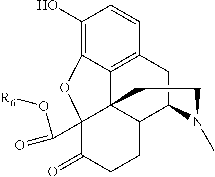 | Hydromorphone |
| 87 | 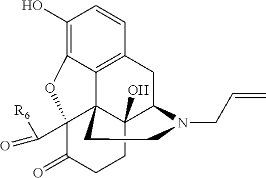 | Naloxone |
| 88 | 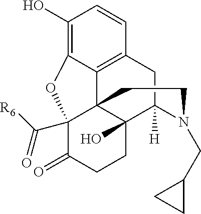 | Naltrexone |
| 89 | 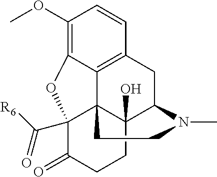 | Oxycodone |
| 90 | 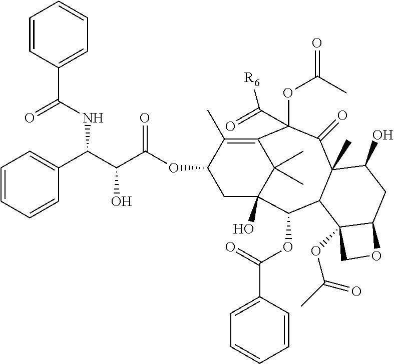 | Paclitaxel |
| 91 | 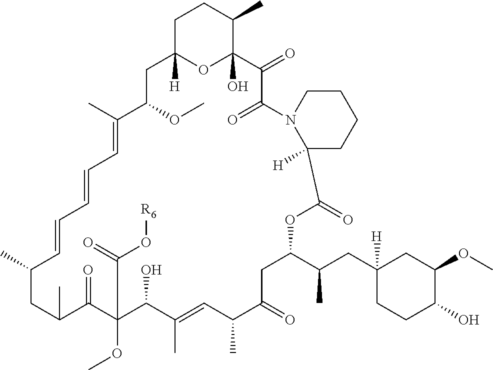 | Sirolimus |
| 92 | 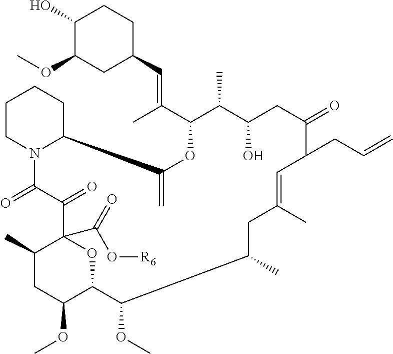 | Tacrolimus |
| 93 | 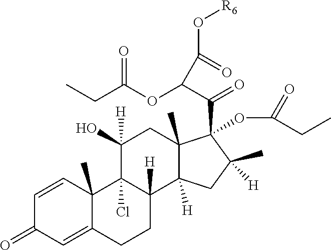 | Beclometasone |
| 94 | 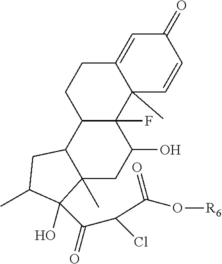 | Clobetasol |
| 95 | 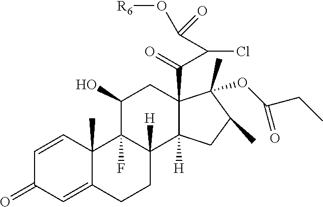 | Clobetasol-17- propionate |
| 96 | 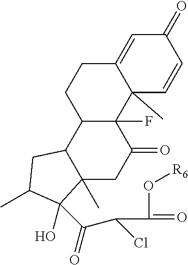 | Clobetasone |
| 97 | 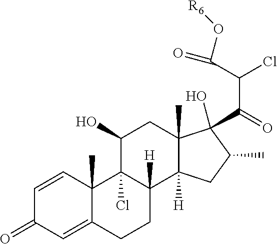 | Mometasone |
| 98 | 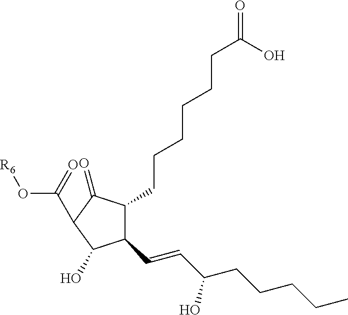 | Alprostadil |
| 99 | 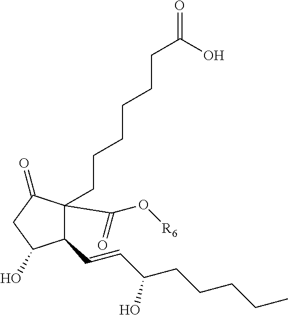 | Alprostadil |
| 100 | 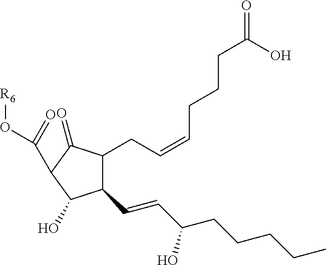 | Dinoprostone |
| 101 | 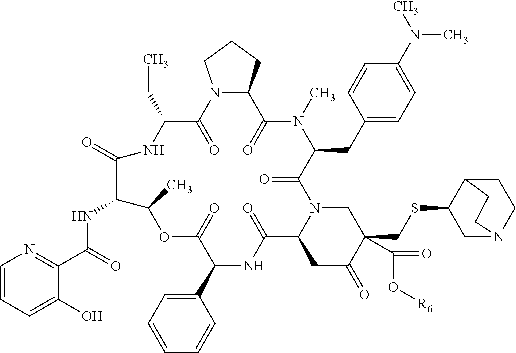 | Quinupristine |
| 102 | 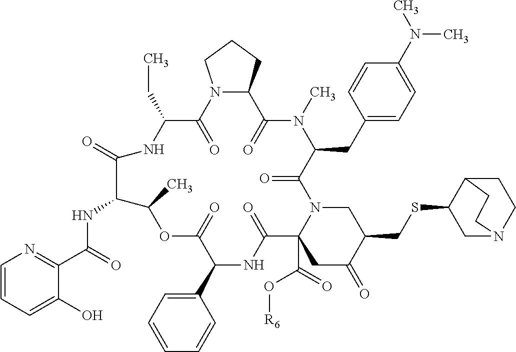 | Quinupristine |
| 103 | 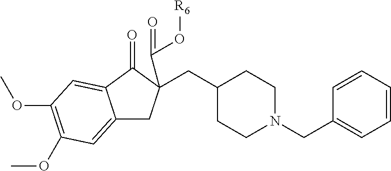 | Donepezil |
| 104 | 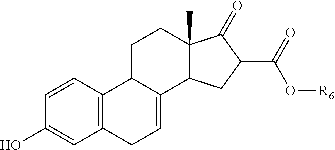 | Equilin |
| 105 | 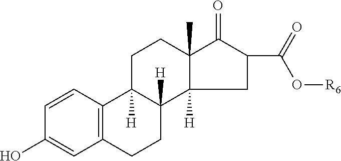 | Estrone |
| 106 | 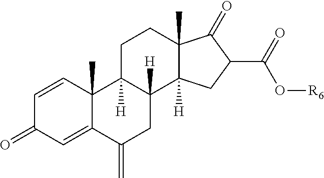 | Exemestane |
| 107 | 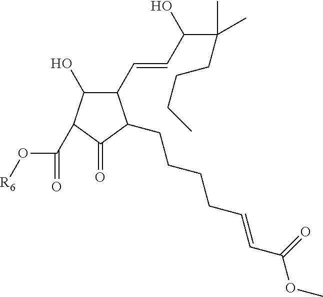 | Gemeprost |
| 108 | 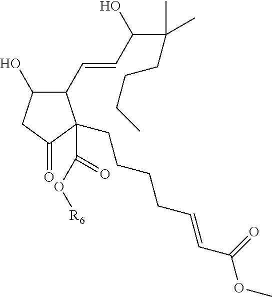 | Gemeprost |
| 109 | 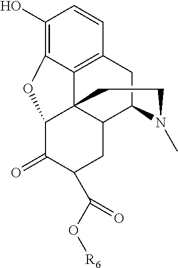 | Hydromorphone |
| 110 | 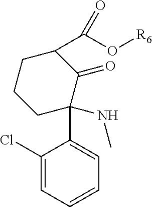 | Ketamine |
| 111 | 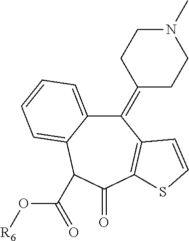 | Ketotifen |
| 112 | 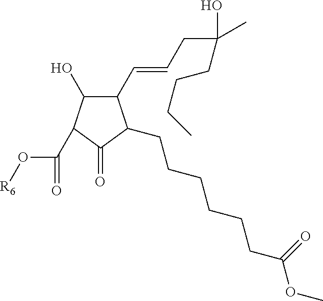 | Misoprostol |
| 113 | 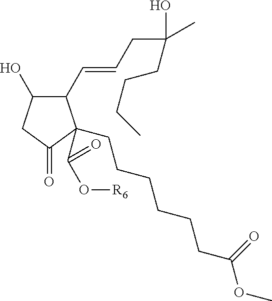 | Misoprostol |
| 114 | 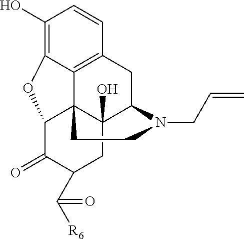 | Naloxone |
| 115 | 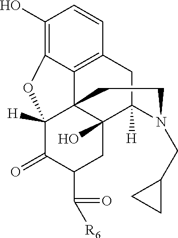 | Naltrexone |
| 116 | 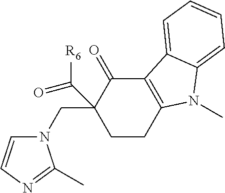 | Ondansetron |
| 117 | 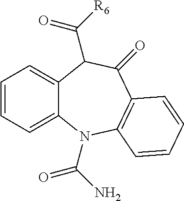 | Oxcarbazepine |
| 118 | 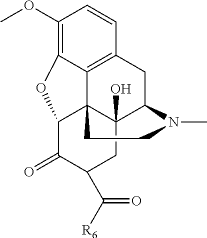 | Oxycodone |
| 119 | 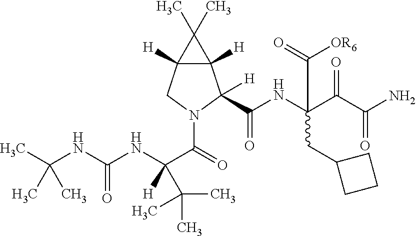 | Boceprevir |
| 120 | 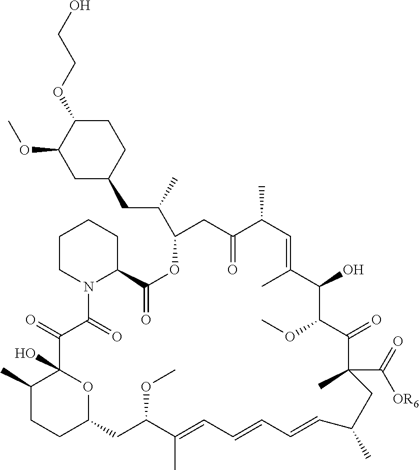 | Everolimus |
| 121 | 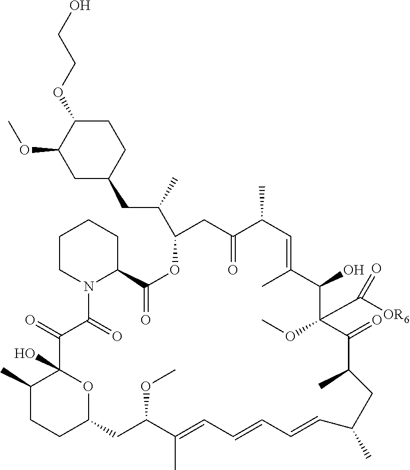 | Everolimus |
| 122 | 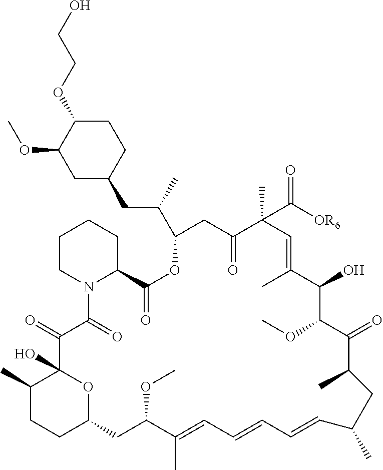 | Everolimus |
| 123 | 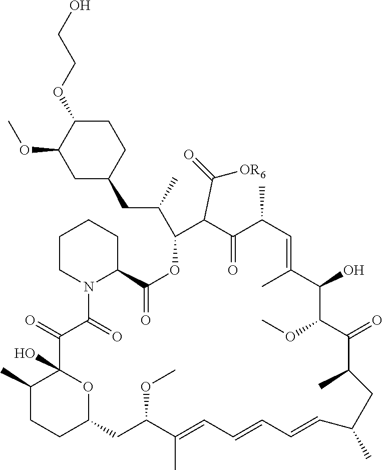 | Everolimus |
| 124 | 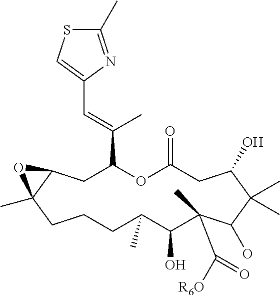 | Patupilone |
| 125 | 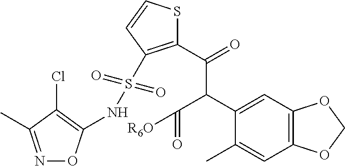 | Sitaxentan |

In an embodiment, the prodrug is selected from a group consisting of the prodrug compounds of Table 1 having Compound Id Nos 1-125. In another embodiment, the prodrug is selected from a group consisting of the prodrug compounds of Table 1 having Compound Id Nos 1-35 and 119-125. In another embodiment, the prodrug is selected from a group consisting of the prodrug compounds of Table 1 having Compound Id Nos 1-35. In another embodiment, the prodrug is selected from a group consisting of the prodrug compounds of Table 1 having Compound Id Nos 1-2, 5-12, 15-28 and 34-35. In another embodiment, the prodrug is selected from a group consisting of the prodrug compounds of Table 1 having Compound Id Nos 22-23. In another embodiment, the prodrug is the prodrug compound of Table 1 having Compound Id No 23. In Table 1, only one selected stereoisomer is presented. Other possible stereoisomeric forms of the compounds in Table 1 are also encompassed by the scope of the present disclosure. Furthermore, in Table 1, only compounds having a single beta-keto carboxylic group are represented. It is contemplated that some of the compounds may be further substituted so as to comprise more than one beta-keto carboxylic group. Such compounds comprising multiple beta-keto carboxylic groups are also encompassed by the scope of the present disclosure.

Many pharmaceutically active agents in use today, and many potential drug candidates, suffer from limited solubility in water. Low solubility of the pharmaceutically active agent makes efficient administration difficult and generally reduces the bioavailability of the pharmaceutically active agent. Prodrugs of the present disclosure, comprising a beta-keto carboxylic acid group or salts or esters thereof, and especially salts thereof, may present an improved solubility in water or aqueous solutions as compared to their corresponding pharmaceutically active agents. Increased solubility in water facilitates administration and uptake of the pharmaceutically active agents. The prodrugs of the present invention may thereby provide an improved bioavailability of the pharmaceutically active agents. Prodrugs of the present disclosure, comprising a beta-keto carboxylic acid group or salts thereof, may be especially useful for improving the aqueous solubility of a poorly soluble pharmaceutically active agent. Prodrugs comprising a beta-keto carboxylic acid group or salts thereof also have the advantage of producing the pharmaceutically active agent without relying on high or low pH values or enzymatic activity. Previous drug derivatives, shown in the prior art, often comprise modifications in the chemical structure of the pharmaceutically active agents, including ester linkages. Such modifications may have the disadvantage of the decomposition route being difficult to predict, since the cleavage of ester bonds require very high or very low pH values or dependent and subject to enzymatic reactions in vivo.

In an embodiment, the pharmaceutically active agent may be a pharmaceutically active agent which has a solubility in water at 25° C. of less than 10 mg/ml, preferably less than 1 mg/ml or less than 0.1 mg/ml or less than 0.01 mg/ml. The pharmaceutically active agent may for example be one of the pharmaceutically active agents listed above, which has a solubility in water at 25° C. of less than 10 mg/ml, preferably less than 1 mg/ml or less than 0.1 mg/ml or less than 0.01 mg/ml.

The prodrug of the present disclosure may also be useful for pharmaceutically active agents that would benefit from sustained release of the pharmaceutically active agent in vivo, since the thermal decomposition of the beta-keto functional group may inherently result in a sustained release of the pharmaceutically active agent upon administration to a subject. Thus, a prodrug of the present disclosure may be useful for a pharmaceutically active agent having a short half-life in vivo.

In an embodiment, the pharmaceutically active agent may be a pharmaceutically active agent which usually requires administration 2 or more times daily in order to maintain a therapeutically effective but non-toxic concentration of the agent in the subject, or which has a half life in vivo of less than 12 hours. In an embodiment of the prodrug, the pharmaceutically active agent, when administered in its conventional form, has a half life in vivo of less than 12 hours. In another embodiment, the pharmaceutically active agent, when administered in its conventional form, has a half life in vivo in the range of 2-9 hours. The pharmaceutically active agent may for example be one of the pharmaceutically active agents listed above, which usually requires administration 2 or more times daily in order to maintain a therapeutically effective but non-toxic concentration of the agent in the subject, or which has a half life in vivo of less than 12 hours.

A prodrug of the present disclosure comprising a beta-keto carboxylic acid salt, may be especially useful for a pharmaceutically active agent which is inherently lipophilic. Such a prodrug may act as an anionic surfactant and form micelles in an aqueous solution. The micelles thus formed would comprise a hydrophilic shell comprising the beta-keto carboxylate groups, and a lipophilic core comprising the pharmaceutically active agent. An example of such a surfactant prodrug is a beta-keto carboxylic acid salt derivative of the pharmaceutically active agent nabumetone.

A physiologically acceptable ester of the prodrug of the present disclosure having a lipophilic R₆ group may be especially useful in a prodrug of a pharmaceutically active agent which is inherently hydrophilic. The amphiphilic nature of such a prodrug may allow the formation of prodrug micelles in aqueous solutions. Micelles of such prodrugs in an aqueous solution would have a hydrophilic shell comprising the hydrophilic pharmaceutically active agent, and a lipophilic core comprising the lipophilic R₆ groups.

In a second aspect thereof, the present disclosure provides a pharmaceutical composition comprising a prodrug as described hereinabove and a pharmaceutically acceptable carrier.

The pharmaceutically acceptable carrier may preferably be selected based on the intended method of administrating the prodrug to a subject. For example, a composition of the prodrug intended for oral administration may preferably comprise a pharmaceutically acceptable carrier suitable for oral formulations and a composition of the prodrug intended for administration by injection may preferably comprise a pharmaceutically acceptable carrier suitable for injectable formulations. Suitable pharmaceutically acceptable carriers may readily be selected by a person skilled in the art of drug formulation.

A prodrug of the present disclosure may provide sustained release of proven pharmaceutically active agents in vivo. Since the release of the pharmaceutically active agent corresponds to the decomposition of the prodrug, i.e. the decarboxylation of the beta-keto carboxylic acid or salt, or the hydrolysis of the beta-keto carboxylic ester and subsequent decarboxylation of the formed beta-keto carboxylic acid or salt, the concentration of the pharmaceutically active agent in a subject will build up gradually. In some cases, it may be desirable to quickly reach a therapeutically effective concentration of the pharmaceutically active agent in the subject. In such cases, the pharmaceutical composition may further comprise an amount of the unmodified form of the pharmaceutically active agent or of another pharmaceutically active agent having the same or similar therapeutic effect as said pharmaceutically active agent. Thus, in an embodiment thereof, the composition of the present disclosure further comprises said pharmaceutically active agent or a second pharmaceutically active agent having the same or similar therapeutic effect as said pharmaceutically active agent. Such a composition will provide a burst effect immediately upon administration to a subject due to the amount of unmodified pharmaceutically active agent, followed by a sustained effect due to the gradual release of the pharmaceutically active agent from the prodrug. In order to limit the number of pharmaceutically active agents administered to the subject, the unmodified pharmaceutically active agent may preferably be the same pharmaceutically active agent as the one the prodrug decomposes into.

The composition of the present disclosure may be presented in different administration forms including, but not limited to, tablets, granules, powders, capsules, solutions, dispersions and suspensions. Different administration forms may be suitable for different modes of administration including, but not limited to oral, parenteral, and intravenous administration.

The prodrugs of the present disclosure decompose in vivo, by decarboxylation of a beta-keto carboxylic acid or salt, or by hydrolysis of the beta-keto carboxylic ester and subsequent decarboxylation of the formed beta-keto carboxylic acid or salt, resulting in the formation of a proven pharmaceutically active agent. In the treatment of disease conditions, it will generally be desirable to maintain a constant level of the pharmaceutically active agent in the subject. Many pharmaceutically active agents suffer from the drawback of low retention time in vivo, e.g. due to rapid degradation and excretion of the formed degradation products. This may necessitate frequent administration of the pharmaceutically active agent, such as two doses per day or more, in order to maintain the concentration of the pharmaceutically active agent in the subject at a therapeutically active level, without exceeding the toxicity level of the pharmaceutically active agent. The severity of potential side effects of a pharmaceutically active agent will generally be related to the dosage. The prodrugs and the compositions described above provide the possibility of maintaining the concentration of a pharmaceutically active agent in a subject at a suitable therapeutically effective level for a prolonged period of time. This may result in improved quality of therapy, reduced side-effects, and improved patient convenience since less frequent administration is required.

Thus, in a third aspect thereof, the present disclosure provides a prodrug or a composition as described hereinabove, for treatment of a mammalian subject suffering from a condition which can be cured or alleviated by administration of said pharmaceutically active agent.

The present disclosure further provides the use of a prodrug or composition as described hereinabove, in the manufacture of a medicament for treatment of a mammalian subject suffering from a condition which can be cured or alleviated by administration of said pharmaceutically active agent.

The prodrugs and compositions described above may be useful in sustained release treatment of conditions and diseases susceptible to treatment with the corresponding unmodified pharmaceutically active agent.

In a fourth aspect thereof, the present disclosure provides the use of a prodrug or a composition as described hereinabove, in the treatment of a mammalian subject suffering from a condition which can be cured or alleviated by administration of said pharmaceutically active agent. In other words, the present disclosure provides a method for treatment of a subject suffering from a condition which can be cured or alleviated by a pharmaceutically active agent, by the administration to said subject of a prodrug of said pharmaceutically active agent according to the first aspect of the present disclosure.

In a fifth aspect thereof, the present disclosure provides a method of preparing a prodrug comprising a beta-keto carboxylic acid, a beta-keto carboxylic acid salt or a beta-keto carboxylic acid ester functional group, said method comprising the steps of:
a) providing a pharmaceutically active agent having the general formula

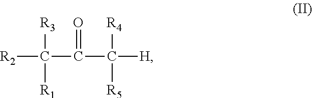

b) mixing said pharmaceutically active agent of step a) with sodium hydride, and a dialkylcarbonate in a suitable solvent and heating the mixture to obtain a prodrug comprising a beta-keto carboxylic acid ester,
c) optionally purifying the prodrug comprising a beta-keto carboxylic acid ester obtained in step b),
d) optionally hydrolyzing the prodrug comprising a beta-keto carboxylic acid ester obtained in step b) or c) to obtain a prodrug comprising a beta-keto carboxylic acid, a beta-keto carboxylic acid salt.

The dialkylcarbonate may preferably be diethylcarbonate. The solvent may preferably be dioxane. In an embodiment, the dialkylcarbonate is diethylcarbonate, and the solvent is dioxane.

The prodrugs of the present disclosure may of course also be prepared by other methods. An example of an alternative method which may be used for preparing the prodrugs of the present disclosure is shown in Tetrahedron Letters, 50, 2009, 104-107 (Tommasi et al.).

Beta-keto carboxylic acids and salts thereof with monovalent cations are generally inherently labile compounds, which are prone to thermal decomposition at room temperature resulting in decarboxylation of the beta-keto carboxylic acid or salt group and the formation of the corresponding ketone. Decomposition occurs spontaneously in the dry state, but is accelerated upon contact with water. Although this decomposition is useful, e.g. for providing controlled release of a pharmaceutically active agent from a prodrug according to the present disclosure, it is often desirable that the decomposition can be suppressed prior to use. The present inventor has surprisingly found that a salt of the prodrug of the present disclosure with a divalent or polyvalent cation presents substantially improved stability as compared to the beta-keto carboxylic acid itself, or monovalent salts thereof.

This method of stabilizing a beta-keto carboxylic acid or a salt thereof with a monovalent cation, using a divalent or polyvalent cation, is not limited to the prodrugs of the present disclosure, but may be applicable to all chemical compounds comprising a beta-keto carboxylic acid or a salt thereof with a monovalent cation. Examples in addition to the above mentioned prodrugs include beta-keto carboxylic acid precursors of fragrance, flavor or aroma molecules. Another example of a class of compounds that can be temporarily stabilized (in the dry state, until dissolved) using the surprising findings above is beta-keto surfactant compounds, as described for example in the published PCT patent application WO2005105963.

Thus, the present disclosure provides a salt of a beta-keto carboxylic acid with a divalent or polyvalent cation.

Any suitable divalent or polyvalent cation may be used, such as a divalent or trivalent metal ion, for example Ca²⁺, Mg²⁺, Zn²⁺, Fe²⁺, Fe³⁺ or Al³⁺ or a mixture thereof. The divalent or polyvalent cation may preferably be a divalent metal cation, such as e.g. Ca²⁺, Mg²⁺, Zn²⁺ or Fe²⁺ or a mixture thereof. The divalent metal ion may preferably be Ca²⁺ or Mg²⁺, more preferably Ca²⁺.

The present disclosure further provides a method of inhibiting decarboxylation of a compound comprising a beta-keto carboxylic acid or a salt thereof with a monovalent cation, characterized in that a dry salt of said beta-keto carboxylic acid with a divalent or polyvalent cation is prepared.

Any suitable divalent or polyvalent cation may be used, such as a divalent or trivalent metal ion, for example Ca²⁺, Mg²⁺, Zn²⁺, Fe²⁺, Fe³⁺ or Al³⁺ or a mixture thereof. The divalent or polyvalent cation may preferably be a divalent metal cation, such as e.g. Ca²⁺, Mg²⁺, Zn²⁺ or Fe²⁺ or a mixture thereof. The divalent metal ion may preferably be Ca²⁺ or Mg²⁺, more preferably Ca²⁺. Preparation of the salt of the beta-keto carboxylic acid with a divalent or polyvalent cation may be performed by any suitable method of salt formation or ion-exchange as readily recognized by a person skilled in the art. Examples include, but are not limited to precipitation, dialysis or the use of suitable ion-exchange media.

In another aspect thereof, the present disclosure provides the novel prodrug compounds listed in column 2 of Table 1, wherein R₆ is as defined above. The novel compounds listed in column 2 of Table 1 are useful as prodrugs for the corresponding pharmaceutically active agents listed in column 3 of Table 1. In an embodiment, the novel prodrug compound is selected from a group consisting of the prodrug compounds of Table 1 having Compound Id Nos 1-125. In another embodiment, the novel prodrug compound is selected from a group consisting of the prodrug compounds of Table 1 having Compound Id Nos 1-35 and 119-125. In another embodiment, the novel prodrug compound is selected from a group consisting of the prodrug compounds of Table 1 having Compound Id Nos 1-35. In another embodiment, the novel prodrug compound is selected from a group consisting of the prodrug compounds of Table 1 having Compound Id Nos 1-2, 5-12, 15-28 and 34-35. In another embodiment, the novel prodrug compound is selected from a group consisting of the prodrug compounds of Table 1 having Compound Id Nos 22-23. In another embodiment, the novel prodrug compound is the prodrug compound of Table 1 having Compound Id No 23. In Table 1, only one selected stereoisomer is presented. Other possible stereoisomeric forms of the compounds in Table 1 are also encompassed by the scope of the present disclosure. Furthermore, in Table 1, only compounds having a single beta-keto carboxylic group are represented. It is contemplated that some of the compounds may be further substituted so as to comprise more than one beta-keto carboxylic group. Such compounds comprising multiple beta-keto carboxylic groups are also encompassed by the scope of the present disclosure.

The R₆ group of each of the novel prodrug compounds of this aspect of the present disclosure may be as defined above in respect of the first aspect of the present disclosure.

A compound comprising a beta-keto carboxy functional group may also be used as a fragrance precursor. In European patent EP 911315, beta-keto esters are mentioned as precursors for fragrance compounds. Beta-keto esters are relatively stable compounds, and release of a fragrance/flavour/aroma ketone from the corresponding beta-keto ester, generally occurs via i) hydrolysis of the ester followed by ii) spontaneous decarboxylation of the formed beta-keto acid, will rely upon the presence of a solvent (often water) acidic or alkaline conditions, and/or on enzymatic or bacterial action to bring about the necessary hydrolysis in step i).

For applications in which such conditions are totally or partially absent, the beta-keto esters are of limited use since hydrolysis will proceed very slowly or not at all. In such applications, e.g. applications wherein a solvent (e.g. water) is absent, wherein pH is more or less neutral, and/or a bacterial-free, or enzyme-free environment/product/ process is required or desired, the desired release rate of the organoleptic compound may not be obtained. Furthermore, beta-keto esters, such as those suggested in EP 911315, are normally oil-like compounds with limited water solubility, which may limit their use in certain aqueous formulations and products.

As a solution to this problem it is provided a precursor for an organoleptic compound, comprising a salt of a beta-keto carboxylic acid with a suitable divalent or polyvalent cation, said compound having the general formula

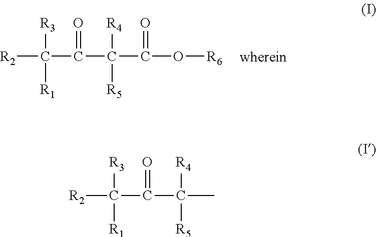

represents a residue of an organoleptic agent having the general formula

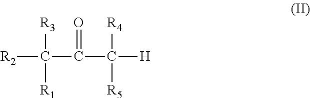

and R₆ represents a divalent or polyvalent cation.

Salts of divalent or polyvalent cations have been shown by the present inventor to be surprisingly stable to decomposition in dry form. When dissolved in water or an aqueous solution, the salt dissociates to the more labile beta-keto carboxylate or carboxylic acid form. Upon dissociation, thermal decomposition of the beta-keto carboxylate or carboxylic acid, and inherently the formation of the ketone functional fragrance compound will be accelerated.

The higher solubility of the beta-keto acid salts, as compared to the lower solubility of the typically oil-like corresponding beta-keto esters, will allow a higher rate of release of the organoleptic compounds compared to when using the corresponding beta-keto ester in the same application.

Any suitable divalent or polyvalent cation may be used. The cation may preferably be a divalent metal cation. More preferably, the cation may be a divalent metal cation selected from the group consisting of Ca²⁺, Mg²⁺, Zn²⁺ or Fe²⁺ or a mixture thereof. The cation may preferably be Ca²⁺.

It has been found that the release rate of the fragrance compound may be tailored by the selection of a suitable cation. For example, sodium salt may be used in order to achieve rapid release of the organoleptic or fragrance compound, while using calcium salt will result in essentially no release at all as long as the salt is kept dry. It is also possible to have a mixture of salts with two or more different cations.

The precursor of the present disclosure has been found to be particularly stable in dry solid form. Thus, in an embodiment, the precursor compound of the present disclosure is preferably a dry solid.

In an embodiment, the organoleptic compound is selected from the group consisting of:
2-heptyl-cyclopentanone,
2,2,6,10-tetramethyltricyclo (6,10)]-undecan-4-one benzylacetone,
1,2,3,5,6,7-hexahydro-1,1,2,3,3-pentamethyl-4H-inden-4-one,
2,5-dimethyl-oct-2-en-6-one,
2-(butan-2-yl)-cyclohexanone,
2-hexyl-cyclopent-2-en-1-one,
2-(1-methylethyl)-5-methyl-cyclohexanone,
2-(2-methylethyl)-5-methyl-cyclohexanone,
3-methyl-cyclopentadecanone,
4-(1,1-dimethylpropyl)pentyl-cyclohexanone,
3-oxo-2-pentyl-cyclopentane-acetic acid methyl ester,
1-(1,2,3,4,5,6,7,8,-octahydro-2,3,8,8-tetramethyl-2-naphthalenyl)-ethanone,
3-methyl-5-propyl-cyclohex-2-en-1-one,
4-(2,6,6-trimethylcyclohex-1-en-1yl)butan-2-one,
4-(2,6,6-trimethylcyclohex-2-en-1-yl)butan-2-one,
2-methyl-5-(1-methylethenyl)-cyclohex-2-en-1-one,
cyclopentadecanone,
1-(4-hydroxyphenyl)-butan-3-one,
4-benzo-1,3-dioxo-5-yl-but-2-one,
4-(1,3-benzodioxol-5-yl)-2-butanone,
nonan-3-one,
nonan-2-one,
octan-2-one,
2-heptanone,
butan-2-one,
6-methyl-hept-5-en-2-one,
6,10-dimethyl-undeca-5,9-dien-2-one,
1-(2,4,4-trimethyl-2-cyclohexen-1-yl)-2-buten-1-one,
carvone,
2-pentyl-cyclopent-2-en-1-one,
3-methyl-2-pentyl-cyclopent-2-en-1-one,
2-hexylidenecyclopentanone,
3,5-diethyl-5,6-dimethyl-2-cyclohexenone,
4,4A,5,6,7,8-hexahydro-6-isopropenyl-4,4A-dimethyl-2(3H)-naphthalenone,
3-methyl-6-propylidenecyclohexanone,
4-(1-methylethyl)cyclohex-2-en-1-one,
(E)-oct-3-en-2-one,
1-(2,3,4,7,8,8A-hexahydro-3,6,8,8-tetramethyl-1H-3A,7-methanoazulen-5-yl)ethanone,
2-hydroxy-3,5-dimethyl-cyclopent-2-en-1-one,
1-(3,3-dimethyl-1-cyclohexen-1-yl)ethanone,
1-(2,4,6-trimethylcyclohex-3-en-1-yl)but-1-en-3-one,
acetylisolongifolene,
2-(3-methylbut-2-en-1-yl)-3-methyl-cyclopent-2-en-1-one,
3-methyl-5-(2,2,3-trimethylcyclopent-3-en-1-yl)pent-3-en-2-one,
5-butylidene-2,2,4-trimethylcyclopentanone,
4,4A,5,6,7,8-hexahydro-6-isopropyl-2(3H)-naphthalenone,
4-(2,6,6-trimethyl-1-cyclohexen-1-yl)-butan-2-one,
4-methoxyphenylethanone,
acetophenone,
1-(2-naphthalenyl)-ethanone,
3-methyl-4-(2,6,6-trimethyl-2-cyclohexen-1-yl)-3-buten-2-one,
2-acetylpyrazine,
3,5,5-trimethyl-cyclohex-2-en-1,4-dione,
(E)-5-methyl-2-hepten-4-one,
dec-3-en-2-one,
2-ethyl-3,6,6-trimethylcyclohex-2-enyl-but-2-en-1-one,
2,4,4,5,5-pentamethyl-1-cyclopenten-1-yl-ethanone,
2-(1-Methylpropyl)-cyclohexanone,
2-Acetonaphtone,
2-Hydroxy-3-methylcyclohex-2-enone,
2-Methyltetrahydrofuran-3-one,
2-Methyltetrahydrothiophen-3-one,
2-octen-4-one,
3-methyl-2,4-nonanedione,
4-Hydroxy-2,5-dimethyl-3(2H)-furanone,
5-Cyclohexadecen-1-one,
Allyl alpha-ionone,
Alpha-Ionone,
Azarbre,
Beta-Ionone,
Cis-Jasmone,
Cosmone,
Decatone,
Furonol acetate,
Gamma-ionone,
Isolongifolanone,
Kephalis,
Mercaptobutanone,
Methyl corylone,
Methyl ionone,
Nectaryl,
Pharaone,
p-Menthane-8-thiol-3-one,
Safraleine,
Spirogalbanone,
2-undecanone, and
Benzylacetone.

Examples of precursors for an organoleptic compound according to the present disclosure include, but are not limited to, those listed in Table 2.

TABLE 2
Precursors for organoleptic compounds and the corresponding
organoleptic compounds.

| Compound | | Organoleptic |
|---|---|---|
| Id No. | Structure | compound |
| 126 | 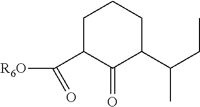 | 2-(1- Methylpropyl) cyclohexanone |
| 127 | 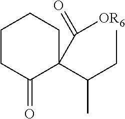 | 2-(1- Methylpropyl) cyclohexanone |
| 128 | 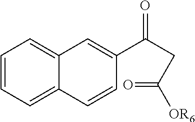 | 2-Acetonaphtone |
| 129 | 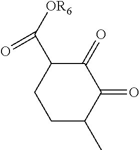 | 2-Hydroxy-3- methylcyclohex-2- enone |
| 130 | 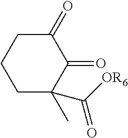 | 2-Hydroxy-3- methylcyclohex-2- enone |
| 131 | 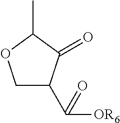 | 2- Methyltetrahydrofuran- 3-one |
| 132 | 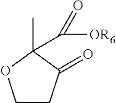 | 2- Methyltetrahydrofuran- 3-one |
| 133 | 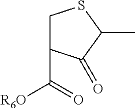 | 2- Methyltetrahydro- thiophen-3-one |
| 134 | 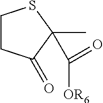 | 2- Methyltetrahydro- thiophen-3-one |
| 135 | 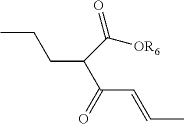 | 2-octen-4-one |
| 136 | 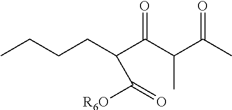 | 3-methy1-2,4- nonanedione |
| 137 | 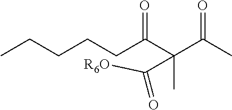 | 3-methy1-2,4- nonanedione |
| 138 | 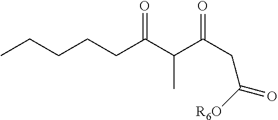 | 3-methy1-2,4- nonanedione |
| 139 | 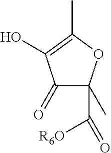 | 4-Hydroxy-2,5- dimethy1-3(2H)- furanone |
| 140 | 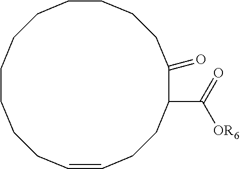 | 5-Cyclohexadecen- 1-one |
| 141 | 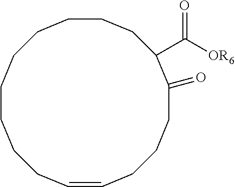 | 5-Cyclohexadecen- 1-one |
| 142 | 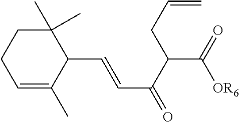 | Allyl alpha-ionone |
| 143 | 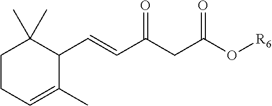 | alpha-Ionone |
| 144 | 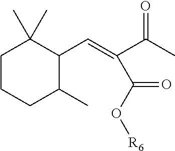 | alpha-Ionone |
| 145 | 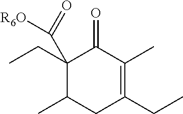 | Azarbre |
| 146 | 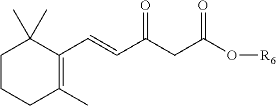 | Beta-Ionone |
| 147 | 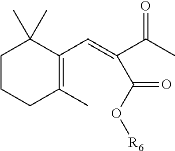 | Beta-Ionone |
| 148 | 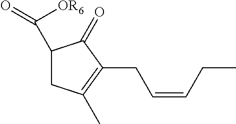 | cis-Jasmone |
| 149 | 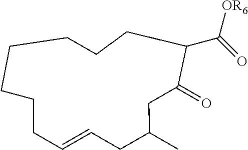 | Cosmone |
| 150 | 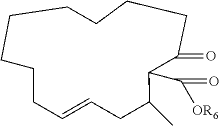 | Cosmone |
| 151 | 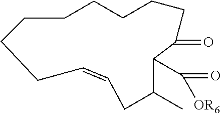 | Cosmone |
| 152 | 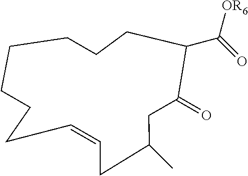 | Cosmone |
| 153 | 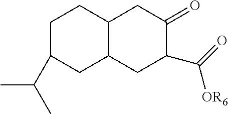 | Decatone |
| 154 | 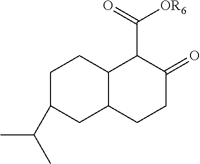 | Decatone |
| 155 | 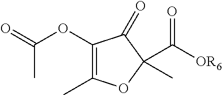 | Furonol acetate |
| 156 | 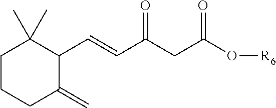 | Gamma-ionone |
| 157 | 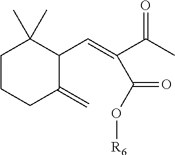 | Gamma-ionone |
| 158 | 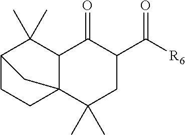 | Isolongifolanone |
| 159 | 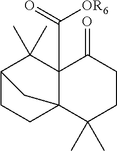 | Isolongifolanone |
| 160 | 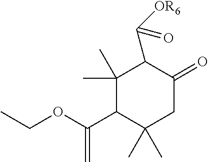 | Kephalis |
| 161 | 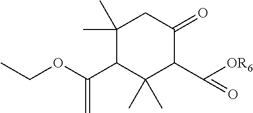 | Kephalis |
| 162 | 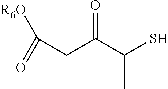 | Mercaptobutanone |
| 163 | 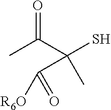 | Mercaptobutanone |
| 164 | 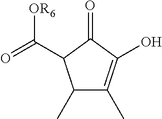 | Methyl corylone |
| 165 | 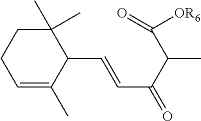 | methyl ionone |
| 166 | 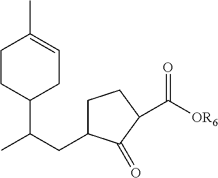 | Nectaryl |
| 167 | 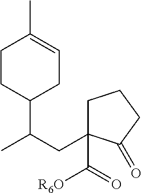 | Nectaryl |
| 168 | 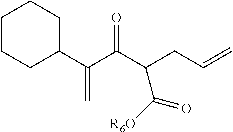 | Pharaone |
| 169 | 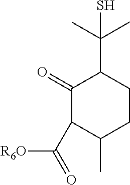 | p-Menthane-8- thiol-3-one |
| 170 | 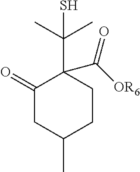 | p-Menthane-8- thiol-3-one |
| 171 | 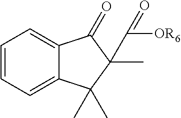 | Safraleine |
| 172 | 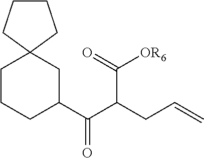 | Spirogalbanone |
| 173 | 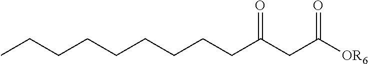 | 2-undecanone |
| 174 | 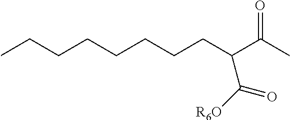 | 2-undecanone |
| 175 | 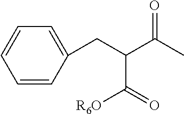 | Benzylacetone |
| 176 | 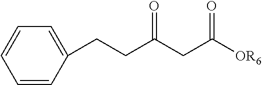 | Benzylacetone |

Stabile salts of beta-keto acids may for example be useful as storage stabile food additives that will release their fragrance or flavor or aroma once they are dissolved in water, e.g. in a soup or hot beverage or other water based food product. Fragrance, flavor and/or aroma release may be accelerated upon heating the product, resulting in a burst effect upon serving the product, which may add value to a consumer product.

Thus the present disclosure provides the use of a precursor for an organoleptic compound according to the present disclosure in the preparation of a foodstuff.

Some of the precursor for organoleptic compounds of the present disclosure may also have other useful properties. For example, the compounds with compound Id No 173 and 174 are useful as precursors for 2-undecanone which, besides being an organoleptic compound, also acts as an insect repellant.

Furthermore, the use of the storage stabile calcium, or other divalent or polyvalent salts of a beta-keto carboxylic acid, e.g. for impregnation of a porous material, such as a textile cloth or a shirt, will allow local and triggered fragrance release to occur when the textile is wetted, e.g. by sweat in the case of a shirt, where release of a odor-masking fragrance may be of value. As soon as the shirt has dried out, the calcium salt is precipitated into its inactive form again, and remaining fragrance remains in the form of the odorless pro-fragrance, for release when needed later. This distinguishes the calcium, and other polyvalent ion salts from the corresponding beta-keto esters; once the esters have been hydrolyzed, e.g. by bacteria or enzymes, the reaction cannot be stopped and started again at will which is an additional technical benefit of the storage stabile calcium salts.

In the same manner, it is possible to deliberately switch on and off the release of the efficient mosquito repellant 2-undecanone (see U.S. Pat No. 7,288,573—“Method of repelling insects”) by impregnation, and subsequent drying, of a cloth or a porous mosquito net with a solution of a calcium salt of compounds 173 and/or 174. When the cloth or net is sprayed with water, the calcium salts dissolve and release of 2-undecanone from the cloth/net is started. When the water has evaporated, the release is stopped, and remaining mosquito repellant is saved for use whenever needed next time.

A compound comprising a beta-keto carboxy functional group may also be used as a decomposing surfactant precursor. This has been described for example in the published PCT patent application WO2005105963 wherein the surfactant precursor is provided in the form of an ester derivative of the beta-keto carboxylic acid, which must be activated by hydrolysis before use. Low storage stability in the dry state of pre-activated (hydrolysed) beta-keto ester precursors has been a technical limitation for the concept of labile beta-keto surfactants.

As a solution to this problem it is provided a surfactant compound, comprising a salt of a beta-keto carboxylic acid with a suitable divalent or polyvalent cation, said compound having the general formula

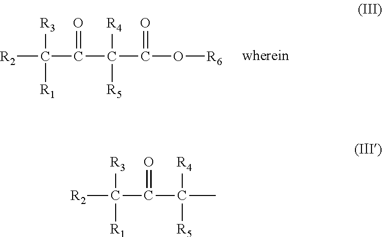

represents the hydrophobic moiety of the surfactant compound
and wherein R₆ represents a divalent or polyvalent cation

Salts of divalent or polyvalent cations have been shown by the present inventor to be surprisingly stable to decomposition in dry form. When dissolved in water or an aqueous solution, the salt dissociates to the more labile beta-keto carboxylate or carboxylic acid form. Upon dissociation, thermal decomposition of the immediately formed beta-keto carboxylate or carboxylic acid surfactant starts, and gradual breakdown of the surfactant via formation of the ketone non-surfactant compound will thereby be initiated/accelerated.

With a surfactant compound comprising a salt of a beta-keto carboxylic acid with a suitable divalent or polyvalent cation, it is possible to store the surfactant, e.g. for use in a laundry detergent, as a dry additive for mixing with a paint formulation just before use, for use in a storage stabile automatic dishwashing powder, or in any application where a temporary emulsifying or detergency action or surface activity will be needed immediately, but after long time of storage on the shelf.

Any suitable divalent or polyvalent cation may be used. The cation may preferably be a divalent metal cation. More preferably, the cation may be a divalent metal cation selected from the group consisting of Ca²⁺, Mg²⁺, Zn²⁺ or Fe²⁺ or a mixture thereof. The cation may preferably be Ca²⁺.

The hydrophobic moiety (III′) comprises at least one hydrophobic group. It is well known to the skilled person that a wide selection of hydrophobic groups exists and that their detailed structures not always are of critical importance in order to achieve surface activity. Thus, any hydrophobic group, not itself susceptible to rapid degradation in aqueous solution, creating an amphiphilic compound when attached to the hydrophilic carboxylate group, may be used in the present surfactant compound. As an example, the hydrophobic group may be a straight-chain, branched-chain or cyclic, saturated or unsaturated, optionally substituted, aliphatic group.

The surfactant compound of the present disclosure has been found to be particularly stable in dry solid form. Thus, in an embodiment, the surfactant compound of the present disclosure is preferably a dry solid.

Examples of surfactant compounds include, but are not limited to, salts of the compounds with compound Id No 173 and 174 with a suitable divalent or polyvalent cation.

When the surfactant salt dissociates/dissolves, e.g. upon contact with water, the free carboxylic acid or carboxylate surfactant formed will gradually (rate being dependent on temperature etc) become a non-surfactant as the beta-keto group decomposes via decarboxylation to form the ketone. As an example, in the case of compound Id No 173 and 174, the non-surfactant ketone formed is 2-undecanone.

### EXAMPLES

### Example 1

### Activation of the Ethyl Benzoyl Acetate Precursor—Preparation of a Sodium Benzoylacetate Solution through Saponification

A 0.2 M solution of ethyl benzoyl acetate (90%, Sigma Aldrich) in 0.5 M NaOH (aq) was prepared at room temperature and stirred for typically 5-24 hours, still at room temperature. After this time a major part of the ethyl benzoyl acetate had been transformed into dissolved sodium benzoyl acetate, as evidenced by monitoring the reaction by analysis of samples by reversed phase HPLC. Some acetophenone and remaining ethyl benzoyl acetate were typically present in the samples, but in minor amounts. The method employed a C8 column with an inner diameter of 150×4.6 mm and 5 micron particles. The mobile phase consisted of 400 ml deionized water+600 ml acetonitrile+0.196 g H₃PO₄ (85%)+2.760 g NaH₂PO₄*H₂O. The flow rate was set to 1.0 mL/min and UV detection at 254 nm was employed for analysis of the compounds ethylbenzoylacetate (precursor), benzoylacetate (model prodrug) and acetophenone (model drug).

### Example 2

### Preparation of Ca(benzoylacetate)₂

A solution of benzoyl acetate, prepared as described in Example 1, was neutralized with diluted HCl to pH 7 and thereafter a large excess of a saturated calcium chloride solution was added, whereby the Ca(benzoylacetate)₂ product precipitated. The solid product was separated from the aqueous solution by filtration, and the sample was allowed to dry at room temperature and ambient humidity. No further purification of the product was performed, due to the finding that the product was readily soluble in water. The absence of a purification step inevitably led to substantial amounts of remaining CaCl₂ in the product (see below).

### Example 3

### Preparation of Benzoylacetic Acid

A solution prepared as described in Example 1 was acidified with diluted HCl to a pH below 2, whereby benzoylacetic acid precipitated. The solid product was separated from the aqueous solution by filtration, the filtrate was washed with a very small portion of distilled water passed through the filter, and the obtained product was thereafter dried without further purification.

### Example 4

### Storage Stability of Activated Bezoylacetate Salts and Their Corresponding Acid

The storage stability of the sodium and calcium salts of benzoylacetate, and of benzoylacetic acid, in solid form, and in aqueous solutions, was investigated by storage of samples under various conditions (see Table 3a above) with regular HPLC- or gravimetric measurements of the samples from time to time. Samples denoted “neutralized” were prepared from a solution prepared as described in Example 1, which was neutralized with diluted HCl after performing the steps in Example 1.

The storage stability of samples evaluated at normal and elevated temperature conditions (60° C.) is summarized in Tables 3a and 3b. From the data it can be concluded that the best storage stability at room temperature is obtained by storage in the form of the calcium salt. The sodium salt requires storage in freezer to achieve long term stability, whereas the solid dry acid showed a half life of about 100 days under typical indoor conditions.

TABLE 3a
Storage stability of sodium benzoylacetate and its corresponding acid at various conditions.

| | | | | | | Alkaline | Neutralized | |
|---|---|---|---|---|---|---|---|---|
| | Solid Na- | Solid Na- | Solid Na- | | | solution of | solution of Na- | |
| | salt, non | salt, | salt, | Solid Na- | Solid Na- | Na-benzoyl | benzoyl | Solid |
| | neutralized, | neutralized, | neutralized, | salt, | salt, | acetate from | acetate from | benzoylacetic |
| Storage | stored at | 30-50% | stored in | neutralized, | neutralized, | Example 1, | Example 1 (pH | acid, at room T |
| time | 30-50% RH | RH at | freezer (ca | stored at | stored at | stored at | 7.1), at room | and ambient |
| (days) | at room T* | room T* | −18° C.)** | 6° C.*** | 60° C.*** | room T* | T* | indoor humidity**** |
| 0 | | | 100 | 103.1 | | | | 100 |
| 0.02 | | | | | 77.2 | | | |
| 0.8 | | | | | 10.7 | | | |
| 2 | 100.0 | 100.0 | 100 | 112.6 | | 100.0 | 100.0 | |
| 5 | | | 124 | 109.2 | | | | |
| 8 | 46.8 | 84.8 | 106 | 102.0 | | 66.2 | 52.0 | |
| 9 | | | | | | | | 94.7 |
| 9.1 | | | | | 3.6 | | | |
| 12 | | | 112 | 100.7 | | | | |
| 16 | | | 106 | 98.5 | | | | |
| 21 | | | 101 | 81.9 | | | | |
| 23 | 12.4 | 33.4 | | | | 10.0 | 8.9 | 87.4 |
| 27 | | | | | | | | 85.4 |
| 29 | | | | | | | | 84.4 |
| 35 | | | | | | | | 81.9 |
| 37 | | | 105 | 74.6 | | | | |
| 42 | | | | | | | | 79.4 |
| 49 | | | 117 | 72.3 | | | | |
| 52 | | | | | | | | 73.6 |
| 185 | | 1.3*** | | | | | | |

*Note: % relative to sample amount remaining after 2 days. By HPLC.
**Note: % relative to initial sample amount. By HPLC.
***Note: % of absolute amount expected to be present in sample. By HPLC.
****Note: weight % of initial amount

TABLE 3b
Storage stability of solid calcium benzoylacetate at various conditions.

| SAMPLE STORAGE HISTORY | | | Absolute amount |
|---|---|---|---|
| Storage time at | Storage | Storage | of calcium |
| room T and | time at | time at | benzoylacetate |
| typical indoor | 50% RH and | 60° C. in | in sample (wt %, |
| humidity (days) | room T (days) | oven (days) | from HPLC)* |
| 1 | 0 | 0 | 35.9 |
| 1.9 | 0 | 0 | 34.2 |
| 8 | 0 | 0 | 43.0 |
| 28 | 0 | 0 | 33.1 |
| 112 | 0 | 0 | 40.4 |
| 9 | 103 | 0 | 32.1 |
| 9 | 0 | 103 | 45.6 |

*Note:
The fact that the samples not even from the beginning contained 100% calcium benzoyl acetate is explained by significant amounts of hygroscopic CaCl₂ remaining from the precipitation (see Example 2). The hygroscopic nature of CaCl₂ explains the lower observed weight-% amount of calcium benzoyl acetate in the samples stored in 50% humidity, and the corresponding higher observed amount in the samples stored at 60° C., relative to the sample stored at indoor temperature and humidity.

### Example 5

### Re-Activation of a Decomposition-Inhibited (Storage Stabile) Beta-Keto Calcium Salt by Dissolution, to Allow Release of the Corresponding Ketone

25 mg of the precipitated and dried calcium salt prepared in Example 2, was, after being subjected to the storage stability test of Example 4, dissolved in 5.66 g of tap water and boiled for two minutes. A strong odour of acetophenone was immediately obtained from the boiling solution, showing that dissolution is an effective way to release the inhibition of the temporarily inhibited beta-keto anion and allow (here thermally induced, deliberately rapid) decomposition into the desired ketone. A similar amount of the dried calcium salt kept in dry form in an identical beaker did not give rise to any acetophenone odour, as expected due to the intrinsic stability of the calcium salt, see Example 4 above.

### Example 6

### Sustained Release of Acetophenone (Model Drug) Demonstrated in-Vitro in a Model Blood Electrolyte Solution at 37° C.

Ethyl benzoyl acetate was saponified according to the description in Example 1, and after 7 hours a solution containing about 0.2 M of the sodium salt of benzoyl acetate was obtained. The solution was diluted 100 times with an aqueous solution containing important blood constituents (inorganic salts/pH buffer system and urea (see Table 4a) in physiologically relevant concentrations. After checking that the pH of the sample was 7.4, the sample was stirred in an oil bath at 37° C. and samples taken for HPLC analysis at regular intervals. The decomposition of the labile beta-keto salt, and the corresponding formation of the model drug acetophenone, is shown in Table 4b. The estimated half-life of the labile beta-keto model prodrug is demonstrated to be about 20 hours under the conditions used, and the example shows that spontaneous, non enzymatic, decomposition of the prodrug, and formation of the desired active drug, occurs on a clinically relevant time scale.

TABLE 4a
Composition of the aqueous model solution of physiologically
important blood solutes, used in Examples 6 and 10 below. The
pH of the solution was adjusted to 7.4 with diluted HCl/NaOH.

| | | Concentration in final |
|---|---|---|
| Substance | Conc (mM) | solution (g/dm³) |
| NaCl | 98.2 | 5.735 |
| KCl | 4.3 | 0.321 |
| NaHCO3 | 20.5 | 1.722 |
| Urea | 4.45 | 0.267 |

TABLE 4b

| | Concentration of sodium | Concentration of |
|---|---|---|
| | benzoyl acetate model | acetophenone (model |
| Time @ 37° C. | prodrug in sample (mM)* | drug) in sample (mM)* |
| 0.42 | 2.66 | 0.16 |
| 2.25 | 2.30 | 0.29 |
| 3.17 | 2.44 | 0.38 |
| 4.17 | 2.40 | 0.48 |
| 6.17 | 1.71 | 0.40 |
| 8.25 | 1.96 | 0.63 |
| 10.17 | 1.69 | 0.73 |
| 12.17 | 1.59 | 0.73 |
| 13.92 | 1.52 | 0.90 |
| 17.92 | 1.43 | 0.92 |
| 21.17 | 1.31 | 1.08 |
| 24.92 | 1.02 | 0.95 |
| 31.33 | 0.94 | 1.11 |
| 48.75 | 0.53 | 1.55 |
| 76.33 | 0.20 | 1.89 |

*HPLC analysis

### Example 7

### Sustained Release of Acetophenone (Model Drug) Demonstrated in Vitro in Pig Blood at 37° C.

50.3 mg of dry Ca(benzoylacetate)₂, prepared as described in Example 2 above, was dissolved in 1000 microliters of distilled water, after having been stored for 128 days at room temperature and normal indoor humidity conditions. The material was taken from the same batch used for the storage stability demonstration in Example 4 (Table 3b) and could therefore be expected to contain about 40 wt % of Ca(benzoylacetate)₂, giving an aqueous solution with an expected concentration of 55 mM of the Ca(benzoylacetate)₂ salt, and a corresponding, twice as high, concentration (110 mM) of the benzoyl acetate ion.

36 microliters of the solution was then added to 2000 μl of cold pig blood (obtained from Scan AB), containing 100-150 mL of a 17% trisodium citrate dehydrate solution per ca 3 litres of blood, as an anti-coagulation additive. The expected benzoylacetate concentration in the blood sample would thus be ca 2 mM which was found to be in good agreement with the measured initial concentration in the sample (see Table 5 below).

The pH of the obtained blood sample was measured, and found to be 6.63, and the sample was thereafter kept in an oil bath at 37° C. with continuous stirring. 20 μl samples were withdrawn from the blood solution at regular intervals, diluted 100 times with mobile phase (as in Example 1) and analysed by HPLC. The results obtained are summarized in Table 5, and it can be seen from the data that breakdown of the benzoyl acetate gave a sustained release of the acetophenone (model drug) over a period of at least 6 hours, with the half life of the benzoyl acetate prodrug being about 3 hours.

TABLE 5

| | Benzoyl acetate ion | Acetophenone |
|---|---|---|
| | concentration | concentration |
| Time at 37° C. | (mM) | (mM) |
| −0.17* | 2.30 | 0.07 |
| 0.25 | 1.83 | 0.09 |
| 1.33 | 1.54 | 0.32 |
| 2.58** | 1.17 | 0.50 |
| 4.75** | 0.929 | 0.866 |
| 5.58 | 0.724 | 0.866 |
| 6.58 | 0.641 | 0.921 |
| 22.58 | 0.0356 | 1.34 |

*Note:
Sample analysed just before heating to 37° C.
**Note:
These two samples were accidentally labelled identically after sampling, and the sampling times of these two sample could therefore, in principle, have been exchanged for one another. However, as seen iif the data is plotted, the trend of the curve obtained from the other samples strongly suggests that the sampling times for the two samples in the above table are correctly connected to the right pair of analysis results.

### Example 8

### Synthesis of ethyl-5-(6-methoxy-2-naphthyl)-beta-keto-pentanoate

Ethyl-5-(6-methoxy-2-naphthyl)-beta-keto-pentanoate, useful as a precursor for the labile beta-keto derivative of Nabumetone (5-(6-methoxy-2-naphthyl)-beta-keto-pentanoate), which may in turn decompose into Nabumetone, was synthesized as follows, using 4-(6-methoxy-2-naphthyl)-2-butanone (Nabumetone) as a starting material. NaH (0.21 g, 8.8 mmol) and diethylcarbonate (1.1 ml, 9.1 mmol) was mixed in dioxane (7.5 ml) and heated for about 30 minutes. Nabumetone (1.04 g, 4.6 mmol) dissolved in dioxane (2.5 ml) was added dropwise. The mixture was refluxed under an argon atmosphere for 3.5 h. The mixture was then cooled and treated with 3M HCl added dropwise to a pH of between 6 and 7. The organic layer was separated from the water phase and the water phase was extracted with diethylether (3*20 ml). The organic layers were combined, dried over MgSO₄, and concentrated in vacuum. The mixture was purified by column chromatography using a packed column of SiO₂ and a range of different elution solvents to increase the separation of the components. The elution started with petroleumether:ethylacetate 15:1, and was then changed to petroleumether:ethylacetate 10:1, and then to petroleumether:ethylacetate. 1:1. By this procedure, five different fractions were separated from the mixture, of which the third gave an NMR fingerprint identical to the target compound ethyl-(5-(6-methoxy-2-naphthyl)-beta-keto-pentanoate: (¹³C NMR (300 MHz, DMSO) 13.97 (CH₃), 38.66 (CH₂) 40.34(CH₂), 43.57(CH₂), 55.10 (CH₃), 60.48 (CH₂) 105.73 (CH), 118.52 (CH) 125.93 (CH) 126.71 (CH), 127.61(CH), 128.54(C),128.78 (CH), 132.79 (C), 135.91 (C), 156.81(C), 167.25 (C), 202.87 (C)). This third fraction was eluted with the petroleumether:ethyl acetate 10:1 solvent, and the NMR spectrum for fraction three also contained three additional peaks, 14.07 (CH3), 20.72 (CH3), 59.71 (CH2) which could represent some remaining ethylacetate.

### Example 9

### Sustained Release of Nabumetone from its Corresponding Beta-Keto Sodium Salt Prodrug, Demonstrated in Phosphate Buffer at Blood pH and 37° C.

20.91 mg of the product synthesised as described in Example 8 (however in this case not purified by the chromatographic step in Example 8) was mixed with 1 ml 0.5 M NaOH at room temperature, whereby a portion of the sample was dissolved. After 30 minutes the mixture was placed in an oven at 37° C., and 10 μl samples was taken at regular intervals, diluted with 1 ml mobile phase (as in Example 1), and analyzed by HPLC, using UV-detection at 240 nm. 3.0 hours after initiation of the saponification reaction, the precursor (retention time 4.6 min) could no longer be detected, and the precursor peak was replaced by a peak from the intermediary beta-keto salt prodrug (retention time 2.3 minutes). At this point in time, a solution of 9 ml H₂O and 0.0762 g NaH₂PO₄ was added to the sample, in order to create a NaH₂PO₄/Na₂HPO₄ pH buffer by neutralization of the NaOH remaining in the sample. After this addition, the pH of the solution was found to be about 7.4. The sample was again placed in the oven at 37° C. and the decarboxylation of the beta-keto salt in solution, gradually giving the commercially available drug Nabumetone, was followed by HPLC by taking 100 μl samples at regular intervals and diluting them with 1 ml mobile phase (see Example 1 above) before injection on the HPLC. The obtained results are shown in Table 6 below. It can be seen from the data that Nabumetone was continuously released into the solution via the spontaneous, non-enzymatic decarboxylation of the dissolved prodrug sodium salt, over a period of more than 120 hours. The half-life of the prodrug was estimated to be about 70 hours in this medium and under the conditions used.

TABLE 6

| | Nabumetone beta- | |
|---|---|---|
| Time after | keto salt prodrug | |
| initiation of | (HPLC area @ | Nabumetone |
| saponification | retention time | (HPLC area @ 3.8 |
| (h) | 2.3 min) | min)* |
| 3.25 | 28341 | 0 |
| 5.1 | 27144 | 0 |
| 21.45 | 22542 | 5265 |
| 24.25 | 24102 | 5388 |
| 24.25 | 21914 | 5496 |
| 28.15 | 19536 | 5804 |
| 45.4 | 18202 | 10083 |
| 50 | 18123 | 11400 |
| 78 | 9528 | 13359 |
| 117 | 9760 | 18156 |
| 194 | 1827 | 22484 |

*Note:
It was verified that the retention time of a sample of commercially purchased pure Nabumetone exactly matched the retention time (3.8 min) ascribed to Nabumetone in the experiment.

### Example 10

In-Vitro Hydrolysis of a Nabumetone Beta-Keto Ester Pro-Prodrug and Sustained Release of Nabumetone Through Subsequent Decarboxylation of the Obtained Beta-Keto Carboxylic Anion, Demonstrated at Physiological Conditions in a Model Blood Electrolyte Solution at Typical Blood pH and 37° C.

14.1 mg of the product synthesized and purified as described in Example 8 was mixed with 375 μL of a 0.5 M solution of KOH in ethanol (manufactured by Merck) and 30 μL of deionised water. The obtained clear yellow solution was frozen ten minutes after preparation, and after thawing the next morning, an additional portion of 20 μL of the ethanolic KOH was added.

After storage at room temperature for 1.5 hours the sample was transferred to an oil bath holding a temperature of 37° C. The sample was thereafter stored 6 hours at this temperature and then cooled to room temperature.

20 μL of the sample was then diluted with 2.0 mL of the model solution of physiologically important blood solutes described in Table 4a above, and placed in a closed vessel in an oil bath at 37° C., with continuous stirring. The sample had a very weakly turbid appearance from start, but the very thin white haze seemed emulsified and stabile, as no sedimentation could be seen when the stirring was turned off temporarily.

20 μl samples were withdrawn at regular intervals, diluted with mobile phase (see Example 1) and analysed by HPLC. The results shown in Table 7.

The pH of the solution was measured after ca 50 h at 37° C., and was found to be 7.5. After about 100-150 hours of storage at 37° C., a microscopic amount of sedimenting crystalline flakes of Nabumetone was detected in the sample. The solubility of Nabumetone in water at 25° C. is 4.7 mg/L (Sepassi et al, Journal of Pharmaceutical Sciences, Vol. 96, No. 10, 2007) and it is therefore not surprising that precipitation of Nabumetone was observed after 100-150 hours, where the theoretical concentration of Nabumetone would be above 40 mg/L, if all formed Nabumetone had been dissolved. The precipitation of Nabumetone led to sampling problems, as sedimentation resulted in the samples collected containing a too low amount of Nabumetone. To obtain a correct value for the final sample, the final HPLC analysis was performed on the full sample, with precipitated crystals and solution altogether quantitatively diluted with mobile phase and analysed. The experiment shows that the beta-keto ester pro-prodrugs can undergo hydrolysis at a physiologically relevant time scale also at moderate pH, similar to the conditions in blood. It further shows that the the beta-keto carboxylic anion prodrug was likely surface active, allowing as much as 40 mg/L of Nabumetone,and a significant amount of the pro-prodrug ester to stay in a stabile dispersion, despite a solubility of Nabmetone of only 4.7 mg/L at room T in water.

TABLE 7

| | Pro-prodrug/ | Prodrug/ | |
|---|---|---|---|
| | beta-keto ester | beta-keto | Nabumetone |
| | (HPLC peak | anion | formed in the |
| Time | area, arbitrary | (HPLC area, | solution |
| (hours) | units) | arbitrary units) | (mg/L)* |
| 0.00 | 1440 | 2055 | 1776 |
| 0.67 | 1368 | 2313 | 1982 |
| 1.67 | 1395 | 2383 | 2000 |
| 2.67 | 1145 | 2266 | 1746 |
| 4.58 | 1130 | 2204 | 1887 |
| 22.6 | 780 | 2079 | 2079 |
| 28.8 | 672 | 2284 | 2492 |
| 48.8 | 618 | 2312 | 2853 |
| 53.6 | 0 | 2350 | 2687 |
| 70.3 | 227 | 2087 | 2807 |
| 70.3 | 245 | 2313 | 2839 |
| 96.8 | 0 | 1915 | No data |
| 96.8 | 80 | 1922 | (2656)* |
| 121.25 | 0 | 1620 | (2715)* |
| 145.0 | 0 | 1444 | (2423)* |
| 172.6 | 0 | 1220 | (3508)* |
| 197.3 | 0 | 1043 | (3240)* |
| 218.4 | 0 | 943 | (3859)* |
| 293.7 | 0 | 400 | (3013)* |
| 335 | 0 | 398.5 | 6301** |

*Note:
Precipitating Nabumetone particles compromised sampling, and the amount of analyzed nabumetone was under-estimated relative to the total amount formed in the sample.
**Note:
This sample was diluted, and analyzed, as a whole, whereby the abovementioned sampling problem was avoided.

### Example 11

### Preparation of a Beta-Keto Calcium Salt Prodrug of Nabumetone, and Demonstration of Storage Stability

12.1 mg of the of ethyl-5-(6-methoxy-2-naphthyl)-beta-keto-pentanoate product prepared in Example 8, purified as described in Example 8, was mixed with 25 mL of a 0.5 M aqueous solution NaOH and homogenised on a vortex mixer. A substantial amount of white crystalline flakes could be seen in the sample, despite the homogenisation. 15 minutes after preparation, the sample was transferred to an oil bath set at 37° C., with continuous gentle magnetic stirring, and kept there for 3.5 hours. Crystalline flakes remained in the sample at the end of the 3.5 hour heat treatment, and the sample was centrifuged and the clear liquid above the sedimented crystals was analysed by HPLC as described above. The sample was found to comprise a very pure solution of the sodium salt obtained by saponification of ethyl-5-(6-methoxy-2-naphthyl)-beta-keto-pentanoate, as evidenced by the characteristic appearance of a strong HPLC peak at 2.3 minutes in the reversed-phase HPLC system.

About ⅛ of the total volume of the above sample, i.e. about 3 ml, was pH adjusted to pH 6.25 with HCl(aq)/NaOH(aq), and thereafter a large excess (several times the volume of the original sample) of saturated CaCl₂ was added, which resulted in the formation of a faint white turbidity, which produced a small white pellet at the bottom of the test tube after centrifugation at 4000 rpm for 5 minutes. After decanting the liquid above the pellet, the test tube with the pellet was left at room temperature for evaporation of the remaining liquid. The sample could contain an approximate maximum of 1.5 mg of product, calculated as Nabumetone beta-keto ester (the pro-prodrug). After four days of storage at room temperature, dry compressed air was blown over the sample for 25 minutes which resulted in a dry and white appearance. The sample was thereafter put in an oven at 60° C. for 17 days to simulate accelerated ageing. After this time, 500 microliters of deionized water was added to the sample followed by vortex mixing for several minutes, resulting in a turbid stabile white emulsion looking much like the one obtained in the initial stage of the experiment in Example 10.

20 μL of this sample was dissolved in 2000 μL of mobile phase, and the solution was analysed by HPLC.

Nabumetone, and its breakdown product (Nabumetone), were observed at the expected retention times in the chromatogram, at similar intensities, see Table 8 below.

TABLE 8

| | Beta keto salt | |
|---|---|---|
| | prodrug | Nabumetone |
| Obtained value (area | 1591 | 1315 |
| units) | | |
| Expected value if no | 55938 | 90 |
| breakdown occured | | |
| during storage and a | | |
| 100% yield was obtained | | |
| upon precipitation with | | |
| calcium chloride | | |
| Relative amount of | 54.7% | 45.3% |
| calcium salt beta-keto | | |
| prodrug/Nabumetone | | |
| remaining after 17 days | | |
| at 60° C.* | | |

*Values recalculated, so as to be comparable with the dilution of the HPLC sample performed above which was 1 + 100 volume units (20 μL + 2000 μL), instead of 1+ 25 as in example 16b (200 μL + 5.0 ml), and taking into account that the precipitated calcium beta-keto prodrug salt was precipitated from ca 3 ml and redissolved in 500 μL of water, giving a factor 6 higher than expected concentration.

The data can be interpreted as follows:
i) The precipitation yield of the calcium beta-keto salt prodrug of Nabumetone above was low, typically only a few percent.
ii) However, after 17 days storage of the dry calcium salt at 60° C., more than 50% of the beta-keto salt prodrug remained in its original form, while the remaining part had slowly broken down to Nabumetone

By using the rule of thumb of doubling of the reaction rate upon increasing the temperature by 10° C., the above data suggests a half life of the Nabumetone prodrug in its calcium salt form of ca 270 days at 20° C.

### Example 12

### Synthesis of Androst-4-ene-2-carboxylic acid, 17-hydroxy-3-oxo-ethyl Ester (a Pro-Prodrug of Testosterone)

Androst-4-ene-2-carboxylic acid, 17-hydroxy-3-oxo-ethyl ester, useful as a pro-prodrug for the labile beta-keto derivative of testosterone which, in turn decomposes controllably into testosterone, was synthesized as follows, using (8R,9S,10R,13S,14S,17S)-17-hydroxy-10,13-dimethyl-1,2,6,7,8,9,11,12,14,15,16,17-dodecahydrocyclopenta[a]phenanthren-3-one (testosterone) as a starting material. NaH (0.17 g, 7.1 mmol) and diethylcarbonate (0.42 ml, 3.5 mmol) was mixed in dioxane (15 ml) and heated for about 30 minutes. At reflux, testosterone (1.0 g, 3.5 mmol) dissolved in dioxane (12 ml), was added. The mixture was refluxed under argon atmosphere for approximately 2.5 hours and then for another 4 hours the following day. The mixture was then cooled and treated with acetic acid to approximately pH 5. The organic phase was separated from the water phase and the water phase was extracted with some additional dioxane. The organic phases were combined and the solution was left to evaporate at room temperature. The oil-like product was analyzed using 13C NMR and found to comprise a mixture of about 1:1 desired product:testosterone+additional dioxane solvent (300 MHz, DMSO, ˜14.9 (CH3 in ethyl group), 53.5 (CH in testosterone), 63.3 (CH2 in ethyl group), 66.4 (dioxane), 79.9 (CH in testosterone) 123.1 (CH), 170.8 (C), 197.9 (C)) and 1H NMR (300 MHz, DMSO, 0.78 (CH3), 1.14 (CH3), 1.19 (CH3 in ethyl group), 3.36 (H2O), 3.56 (dioxane), 4.07 and 4.09 (CH2 in ethyl group).

### Example 13a

### Demonstration of the Higher Aqueous Solubility of a Beta-Keto Salt Compared to the Corresponding Ketone to Which it Decomposes

The solubility of sodium benzoyl acetate was shown to be above 200 mM as saponification according to Example 1 (with or without neutralization to pH 7 with HCl) produced clear solutions. Moreover a solubility test, where 58.3 mg of dry calcium benzoyl acetate, prepared as in Example 2, was mixed with increasing amounts of water until full dissolution was obtained, showed that 0.92 ml water dissolved all but a very insignificant part of the 58.3 mg powder. Thus the solubility limit for the calcium salt was determined to be close to 63 mg/ml. Since the calcium salt sample which had been kept at room temperature since preparation, contained only 40 wt % of the calcium benzoyl acetate (see Example 4b), this solubility corresponds to a concentration of 69 mM Ca(benzoylacetate)₂ and 138 mM benzoylacetate in the solution. In comparison with the tabulated solubility for acetophenone (46 mM), it is clear that both the calcium benzoylacetate and the corresponding sodium salt allow a higher total concentration of acetophenone to be obtained in a given aqueous system. The results are summarized in table 9a below.

TABLE 9a

| | | Solubility | Increase in |
|---|---|---|---|
| | | (mM, as | solubility |
| | Solubility | acetophenone | compared to |
| Substance | (mM) | equivalents) | acetophenone |
| Acetophenone | 46 | 46 | |
| Sodium benzoylacetate | ≧200 | ≧200 | ≧4.3 x |
| Calcium(benzoylacetate)₂ | 69 | 138 | 3 x |

### Example 13b

### Demonstration of Higher Aqueous solubility of a Beta-Keto Salt as Compared to the Corresponding Ketone to Which it Decomposes

12.1 mg of the of ethyl-5-(6-methoxy-2-naphthyl)-beta-keto-pentanoate product prepared and purified as in Example 8 was mixed with 25 mL of a 0.5 M aqueous solution NaOH and homogenised on a vortex mixer. A substantial amount of white crystalline flakes were observed in the sample, despite the homogenisation. 15 minutes after preparation of the sample, the sample was transferred to an oil bath set at 37° C. with continuous gentle magnetic stirring, and kept there for 3.5 hours. Crystalline flakes remained in the sample at the end of the 3.5 hours heat-treatment, and the sample was thereafter centrifuged and the clear liquid above the sedimented crystals was analysed by HPLC as described above. It was found that the sample comprised a very pure solution of the sodium salt obtained by saponification of ethyl-5-(6-methoxy-2-naphthyl)-beta-keto-pentanoate, as evidenced by the characteristic appearance of a strong HPLC peak at 2.3 minutes in the reversed-phase HPLC system. The sample showed the following HPLC areas.

TABLE 9b

| Area at | Area at | Area at retention |
|---|---|---|
| retention | retention | time 4.6 minutes |
| time 2.3 minutes | time 3.8 minutes | characteristic for |
| characteristic for | characteristic for | detection of the |
| detection of the | detection of | beta-keto ester |
| beta-keto anion. | Nabumetone | pro-prodrug |
| (arbitrary units)* | (arbitrary units)* | (arbitrary units)* |
| 36218 | 59 | 632 |

*Note:
All units are the same, and represent a directly comparable measure of the relative peak areas in the chromatogram. 200 μL of sample was diluted with 5.0 ml of mobile phase and pH adjusted to pH 3, before injection on the HPLC column.

After storage around one hour at room temperature a fraction of the sample was then filtered twice through a 0.2 μm filter (Whatman, 0.2 μm PS) fitted to a syringe to give a clear solution. 200 μL of this filtered solution was transferred, in a tightly closed vessel, to an oil bath at 95° C. and kept at this temperature for about 5 hours to bring about quantitative decarboxylation of the labile, dissolved beta-keto carboxylic salt prodrug derivative of Nabumetone.

The heated sample was then mixed with 35 mL of mobile phase (see Example 1) whereby a few solid crystals (comprising Nabumetone) was seen to not dissolve. In order to assure a correct sampling for HPLC analysis, 5 mL of the 35 mL sample was filtered through a 0.2 μm filter, after the whole 35 mL sample had been pH adjusted to 4.21 by 300 μL 0.1 M HCl+100 μL 1 M HCl, to assure that the pH of the sample was close to the pH of the mobile phase itself (pH 4.30). The filtered solution was thereafter analysed by HPLC and found to contain 0.777 mg Nabumetone per liter. If recalculated to the corresponding concentration of nabumetone equivalents in the form of the beta-keto salt prodrug present in dissolved form in the filtered 200 μl sample that was subsequently heat-treated to form Nabumetone above, the obtained concentration corresponds to a Nabumetone concentration of 136.7 mg Nabumetone per liter in the clear aqueous solution, at room temperature, before the heat-treatment at 95° C. As the literature value for the solubility of Nabumetone in water at 25° C. is 4.7 mg/L (Sepassi et al, Journal of Pharmaceutical Sciences, Vol. 96, No. 10, 2007) it can be concluded that the solubility of the, normally very poorly soluble, drug Nabumetone could be increased at least 29 times, by conversion into the corresponding sodium beta-keto carboxylic salt as shown above.

TABLE 9b2

| | | | Increase in solubility |
|---|---|---|---|
| | | | compared to acetophenone |
| | | | (number of times higher |
| | | Solubility | total concentration of |
| | Solubility | (mg/L, as | Nabumetone obtainable in the |
| Substance | (mg/L) | Nabumetone equivalents) | system using the prodrug) |
| Nabumetone | 4.7 | | |
| Sodium beta-keto salt | | 136.7 | 29 X |
| prodrug of Nabumetone | | | |

### Example 13c

### Demonstration of the Higher Aqueous Solubility of a Beta-Keto Salt Compared to the Corresponding Ketone to Which it Decomposes

19 mg of the product obtained in Example 12 was mixed with 200 μL of 0.5 M KOH in ethanol+18 μL of deionised water. After 16 minutes at room temperature, 100 μL of the sample was evaporated to dryness by blowing of compressed air over the sample, and after adding of 3000 μL of deionised water, a turbid mixture giving a stabile foam upon shaking was obtained. The mixture was neutralized by 220 μL of 0.1 M HCl to pH 7.6 and thereafter filtered through a 0.2 μm filter. 20 μL of the clear filtrate was mixed with 2000 μL of mobile phase and analysed by HPLC at λ=244 nm. A strong peak of 5277 area units was obtained at 2.78 minutes retention time. Using a pure testosterone reference sample, which also produced a peak at this retention time, the testosterone concentration was determined to be 0.18 mg testosterone per ml in the filtrate before dilution in the mobile phase. As the literature gives a solubility (at 37° C.) for testosterone of 0.039 mg/ml (Okimotoa et al., Journal of Controlled Release, Vol. 58, Issue 1, 1999, Pages 29-38) it may be concluded that i) the obtained peak at 2.78 minutes could not be ascribed to any major extent to testosterone and ii) that the ethanolic alkaline saponification of the beta-keto ester pro-prodrug of testosterone produced a surface active prodrug (deduced from the observation of a foaming solution) in the form of the corresponding beta-keto potassium salt, having a solubility of at least 0.18 mg/ml, i.e. at least 4.6 times that of testosterone, showing up at the same retention time as testosterone in the chromatogram. The above estimation of the minimum solubility of the testosterone prodrug salt is based on the assumption that extinction coefficients at λ=244 nm are similar for our beta-keto salt and testosterone, which was confirmed by spectrophotometric analysis of the two compounds diluted in ethanol.

1-35. (canceled)

36. A prodrug having the general formula (I): 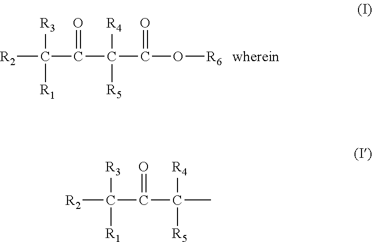
represents a residue of a pharmaceutically active agent having the general formula 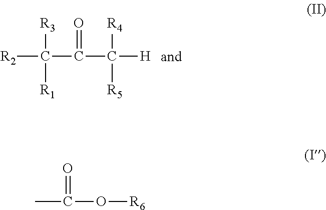
represents —COOH, a salt of —COOH with a physiologically acceptable cation, or an ester of —COOH,
for use in therapy.

37. A prodrug according to claim 36, wherein formula (I″) represents —COOH or a salt of —COOH with a physiologically acceptable cation.

38. A prodrug according to claim 37, wherein formula (I″) represents a salt of —COOH with a physiologically acceptable cation and said cation is Ca²⁺ or Mg²⁺.

39. A prodrug according to claim 36, wherein said prodrug is in dry solid form.

40. A prodrug according to claim 36, wherein said pharmaceutically active agent in its conventional form has a solubility of less than 10 mg/ml in water at 25° C.

41. A prodrug according to claim 36, wherein said pharmaceutically active agent, when administered in its conventional form, usually requires administration 2 or more times daily in order to maintain a therapeutically effective amount of the agent in the subject.

42. A prodrug according to claim 36, wherein said pharmaceutically active agent is selected from the group consisting of Alclometasone, Alprostadil, Beclometasone, Betamethasone, Boceprevir, Budesonide, Bupropion, Camphor, Clarithromycine, Clobetasol, Clobetasone, Cortisone, Cyproterone, Daunomycin, Desonide, Desoximetasone, Dexamethasone, Dinoprostone, Docetaxel, Donepezil, Doxorubicin, Droperidol, Dydrogesterone, Ebastine, Epirubicin, Equilin, Erythromycin, Estrone, Etonogestrel, Everolimus, Exemestane, Fludrocortisone, Flumetasone, Fluocinolone acetonide, Fluprednidene, Gemeprost, Haloperidol, Hydrocortisone, Hydromorphone, Idarubicin, Ketamine, Ketobemidone, Ketotifen, Levo Norgestrel, Lofepramine, Medroxyprogesterone, Megestrol, Melperone, Methadone, Methylprednisolone, Mifepristone, Misoprostol, Mometasone, Nabumetone, Naloxone, Naltrexone, Nandrolone, Nomegestrol, Norethisterone, Ondansetron, Oxcarbazepine, Oxycodone, Paclitaxel, Patupilone, Pentoxifylline, Prednisolone, Prednisone, Progesterone, Propafenone, Propiomazine, Quinupristine, Rimexolone, Sirolimus, Sitaxentan, Spironolactone, Tacrolimus, Testosterone, Tibolone, Triamcinolone, Trimegestone, and Warfarin.

43. A prodrug according to claim 36, wherein said pharmaceutically active agent is selected from the group consisting of Alclometasone, Camphor, Clarithromycine, Clobetasone, Cyproterone, Daunomycin, Desoximetasone, Droperidol, Dydrogesterone, Ebastine, Erythromycin, Haloperidol, Idarubicin, Ketobemidone, Medroxyprogesterone, Megestrol, Melperone, Methadone, Nabumetone, Pentoxifylline, Progesterone, Propafenone, Propiomazine, Rimexolone, Sirolimus, Tacrolimus, Warfarin, Boceprevir, Everolimus, Patupilone, and Sitaxentan.

44. A prodrug according to claim 36, wherein said pharmaceutically active agent is selected from the group consisting of Alclometasone, Camphor, Clarithromycine, Clobetasone, Cyproterone, Daunomycin, Desoximetasone, Droperidol, Dydrogesterone, Ebastine, Erythromycin, Haloperidol, Idarubicin, Ketobemidone, Medroxyprogesterone, Megestrol, Melperone, Methadone, Nabumetone, Pentoxifylline, Progesterone, Propafenone, Propiomazine, Rimexolone, Sirolimus, Tacrolimus, and Warfarin.

45. A prodrug according to claim 36, wherein said pharmaceutically active agent is selected from the group consisting of Alclometasone, Camphor, Clobetasone, Cyproterone, Daunomycin, Desoximetasone, Droperidol, Dydrogesterone, Ebastine, Haloperidol, Idarubicin, Ketobemidone, Medroxyprogesterone, Megestrol, Melperone, Methadone, Nabumetone, Pentoxifylline, Progesterone, Propafenone, Propiomazine, Rimexolone, Tacrolimus, and Warfarin.

46. A prodrug according to claim 36, wherein said pharmaceutically active agent is Nabumetone.

47. A pharmaceutical composition comprising a prodrug according to claim 36, and a pharmaceutically acceptable carrier.

48. A pharmaceutical composition according to claim 47, further comprising said pharmaceutically active agent or a second pharmaceutically active agent having the same or similar therapeutic effect as said pharmaceutically active agent.

49. A prodrug according to claim 36, for treatment of a mammalian subject suffering from a condition which can be cured or alleviated by administration of said pharmaceutically active agent.

50. A method for the treatment of a mammalian subject comprising administering an effective amount of a prodrug according to claim 36 to treat a condition which can be cured or alleviated by such administration.

51. A method for the treatment of a mammalian subject comprising administering an effective amount of a prodrug according to claim 37 to treat a condition which can be cured or alleviated by such administration.

52. Method of inhibiting decarboxylation of a compound comprising a beta-keto carboxylic acid or a salt thereof with a monovalent cation, characterized in that a dry salt of said beta-keto carboxylic acid with a divalent or polyvalent cation is prepared.

53. Method according to claim 52, wherein said divalent or polyvalent cation is a divalent metal cation.

54. Method according to claim 53, wherein said divalent or polyvalent cation is Ca²⁺, Mg²⁺, Zn²⁺ or Fe²⁺.

55. Method according to claim 54, wherein said divalent or polyvalent cation is Ca²⁺.
